(19) United States
(12) Patent Application Publication
(10) Pub. No.: US 2026/0218245 A1
(43) Pub. Date: Jul. 30, 2026

(54) RECOMBINANT PRODUCTION OF ZIZAENE AND OTHER SESQUITERPENES FORMED BY CONVERSION OF THE BISABOLYL CATION

(71) Applicant: ISOBIONICS B.V., Geleen (NL)

(72) Inventors: Janani DURAIRAJ, Binningen (CH); Aalt-Jan VAN DIJK, Wageningen (NL); Thamara HESSELINK, Wageningen (NL); Martinus Julius BEEKWILDER, Geleen (NL); Elena MELILLO, Geleen (NL)

(21) Appl. No.: 19/141,368

(22) PCT Filed: Dec. 18, 2023
(86) PCT No.: PCT/EP2023/086229

§ 371 Date: Jun. 20, 2025

(30) Foreign Application Priority Data
Dec. 22, 2022 (EP) 22215964.2

(51) Int. Cl. C12P 5/00; C12N 9/88; C12N 15/70; C12N 15/74; C12P 15/00

(52) U.S. Cl. CPC C12P 5/007; C12N 9/88; C12N 15/70; C12N 15/74; C12P 15/00

(57) ABSTRACT

The present invention refers to a method for producing a zizaene-containing composition comprising the following subsequent steps: i) providing farnesyl pyrophosphate and at least one polypeptide exhibiting zizaene synthase activity under conditions suitable for the polypeptide to convert at least a portion of the farnesyl pyrophosphate into zizaene; ii) converting, by means of the polypeptide, at least a portion of the farnesyl pyrophosphate into a bisabolyl cation; and iii) producing a zizaene-containing composition by converting, by means of the polypeptide, the bisabolyl cation into zizaene and, optionally, at least one further compound, wherein the at least one polypeptide comprises an amino acid sequence selected from the group consisting of: a) an amino acid sequence as shown in SEQ ID NO: 1; b) an amino acid sequence which is at least 55% identical to the amino acid sequences as shown in SEQ ID NO: 1; c) an amino acid sequence encoded by a nucleic acid sequence as shown in any one of SEQ ID NOs: 2, 3 or 5; d) an amino acid sequence encoded by a nucleic acid sequence which is at least 55% identical to a nucleic acid sequence as shown in any one of SEQ ID NOs: 2, 3 or 5; and e) an amino acid sequence being a fragment of any one of the sequences listed in a) to d). Moreover, the invention is, inter alia, directed to a zizaene-containing composition obtainable with said method as well as to an according non-human host cell and an according polypeptide exhibiting zizaene synthase activity.

### TECHNICAL FIELD

The present invention concerns the field of recombinant production of zizaene and other sesquiterpenes, formed by conversion of the bisabolyl cation, and related compounds. In particular, it refers to a method for producing a zizaene-containing composition, wherein farnesyl pyrophosphate is converted into zizaene by at least one polypeptide exhibiting zizaene synthase activity as well as to a composition obtainable with said method. Moreover, the invention relates to a method for producing a composition comprising at least one oxidation product of zizaene as well as to a corresponding non-human host cell or transgenic organism and an according polypeptide exhibiting zizaene synthase activity.

### INTRODUCTION

Vetiver oil is used in fine fragrances for long-lasting, precious wood notes. Vetiver oil is extracted from vetiver grass (Vetiveria zizanoides), which is mainly produced in Indonesia, and contains more than 150 sesquiterpene-derived constituents. Vetiver grass harvesting is aimed at collecting the roots which go deep in the soil. Hence, it is very labor intensive and leads to a lot of erosion—rendering an increasing need for vetiver oil alternatives.

Historically, there has been some debate on the key organoleptic components of vetiver oil. Nowadays however, the literature generally agrees on the importance of zizaene (cf. Form. I below; CAS: 18444-94-5) as well as of its oxidation products zizanal (cf. Form. II; CAS: 82509-29-3), khusimol (cf. Form. III; CAS: 16223-63-5) and khusimone (cf. Form. IV; CAS: 30557-76-7) for the properties of vetiver oil (Ouyang Angew. Chem. Int. Ed. 2021, 60, 5666-5672; Sell—A Fragrant Introduction to Terpenoid Chemistry; Panten—Flavors and Fragrances, 4. Natural Raw Materials).

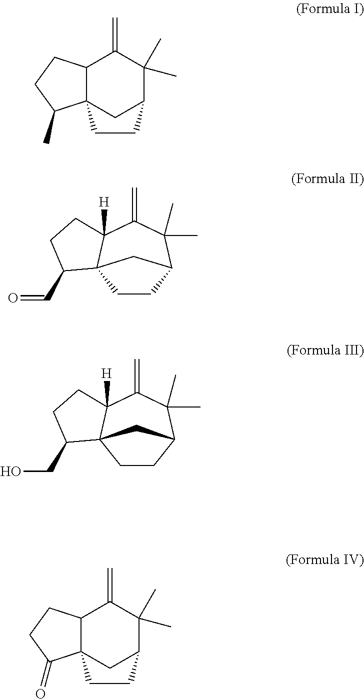

For a biotechnologically produced alternative to vetiver oil, enzymes for producing the key organoleptic components, in particular zizaene, would be needed. However, the only zizaene synthase, which has been known until now, is the one from vetiver (VzZIS protein AJL25242.1, cf. WO 2010/134004 A1 of Firmenich SA).

The zizaene synthase from vetiver does not only produce zizaene from farnesyl pyrophosphate but also specific minor products in specific ratios, wherein the minor products and their oxidation products have an impact on the organoleptic properties of vetiver oil as well.

For the use in fine fragrances, it may be desirable, however, to have a zizaene synthase at hand, which produce different ratios of minor products or even different minor products—rendering a composition with modified organoleptic properties.

### DESCRIPTION OF INVENTION

Therefore, the problem underlying the present invention was to identify an alternative zizaene synthase, which is suitable for the recombinant production of zizaene and related products, and, preferably, produces a zizaene-containing composition having a different organoleptic profile compared to one obtained with the known zizaene synthase from vetiver.

The problem was surprisingly solved by a method for producing a zizaene-containing composition, which comprises the following subsequent steps:
i) providing farnesyl pyrophosphate and at least one polypeptide exhibiting zizaene synthase activity under conditions suitable for the polypeptide to convert at least a portion of the farnesyl pyrophosphate into zizaene;
ii) converting, by means of the polypeptide, at least a portion of the farnesyl pyrophosphate into bisabolyl cations;
iii) producing a zizaene-containing composition by converting, by means of the polypeptide, the bisabolyl cations into zizaene and, optionally, at least one further compound; and
iv) optionally removing, at least to a large extent, any remaining farnesyl pyrophosphate and/or the at least one polypeptide exhibiting zizaene synthase activity from the zizaene-containing composition,
wherein the at least one polypeptide comprises or consists of 1) an amino acid sequence selected from the group consisting of:
a.1) an amino acid sequence as shown in SEQ ID NO: 1;
b.1) an amino acid sequence which is at least 55%, preferably at least 60%, more preferably at least 65%, at least 70%, at least 75%, at least 80% or at least 85%, even more preferably at least 90%, at least 91%, at least 92%, at least 93% or at least 94% and most preferably at least 95%, at least 96%, at least 97%, at least 98% or at least 99% identical to the amino acid sequences as shown in SEQ ID NO: 1;
c.1) an amino acid sequence encoded by a nucleic acid sequence as shown in any one of SEQ ID NOs: 2, 3 or 5;
d.1) an amino acid sequence encoded by a nucleic acid sequence which is at least 55%, preferably at least 60%, more preferably at least 65%, at least 70%, at least 75%, at least 80% or at least 85%, even more preferably at least 90%, at least 91%, at least 92%, at least 93% or at least 94% and most preferably at least 95%, at least 96%, at least 97%, at least 98% or at least 99% identical to a nucleic acid sequence as shown in any one of SEQ ID NOs: 2, 3 or 5; and
e.1) an amino acid sequence being a fragment of any one of the sequences listed in a.1) to
d.1), said fragment exhibiting zizaene synthase activity;
or 2) an amino acid sequence selected from the group consisting of:
a.2) an amino acid sequence as shown in any one of SEQ ID NOs: 7 to 28, preferably 7 to 24 or 28;
b.2) an amino acid sequence which is at least 55%, preferably at least 60%, more preferably at least 65%, at least 70%, at least 75%, at least 80% or at least 85%, even more preferably at least 90%, at least 91%, at least 92%, at least 93% or at least 94% and most preferably at least 95%, at least 96%, at least 97%, at least 98% or at least 99% identical to an amino acid sequence as shown in any one of SEQ ID NOs: 7 to 28, preferably 7 to 24 or 28; and
c.2) an amino acid sequence being a fragment of any one of the sequences listed in a.2) and b.2), said fragment exhibiting zizaene synthase activity.

### Definitions

The term “zizaene-containing composition” as used herein is defined as zizaene or, preferably, a mixture of zizaene and one or more other terpene and/or terpenoid formed by conversion of the bisabolyl cation (cf. Form. VII), wherein zizaene or the mixture, respectively, may be solved or dispersed in an according medium and/or mixed with other components. Preferably, the zizaene-containing composition comprises zizaene and at least one further compound selected from the group consisting of α-funebrene, unknown terpene X, β-funebrene, unknown terpene Y, prezizaene, cis-muurola-4(15),5-diene, β-acoradiene, α-neocallitropsene and acorenol.

Herein, the term “polypeptide” is defined as a contiguous sequence of amino acids linked to each other by peptide bounds. A polypeptide according to the present invention, typically, comprises at least 50, at least 100 or at least 200 amino acid residues in length such that the chain of amino acids can form a three-dimensional structure required to exert zizaene synthase activity. The term “protein” is used interchangeably herein.

The formulations “a (or the) polypeptide exhibiting zizaene synthase activity” and “at least one (or the at least one) polypeptide exhibiting zizaene synthase activity” are used synonymously herein.

The term “zizaene synthase activity” as used herein is defined as follows:

A polypeptide exhibiting such an activity has the ability to convert at least a portion of the provided farnesyl pyrophosphate into zizaene (cf. Form. I above) and preferably belongs to the enzyme class (EC) 4.2.3.X, for example to EC 4.2.3.37 or EC 4.2.3.B17.

Preferably, an according polypeptide converts farnesyl pyrophosphate—in at least one side reaction—into at least one, preferably at least two further compounds (sesquiterpenes or sesquiterpenoids other than zizaene) selected from the group consisting of α-funebrene, unknown terpene X, β-funebrene, unknown terpene Y, prezizaene, cis-muurola-4(15),5-diene, β-acoradiene, α-neocallitropsene and acorenol, preferably consisting of α-funebrene, β-funebrene, unknown terpene Y and β-acoradiene, more preferably consisting of α-funebrene and β-acoradiene.

As in case of zizaene, said further compounds are formed by conversion of the bisabolyl cation (cf. Form. VII), wherein the bisabolyl cation is formed by isomerization of the nerolidyl cation (cf. Form. VI) which, in turn, is formed by isomerization of the farnesyl cation (cf. Form. V) being formed by cleavage of pyrophosphate in the reactive center of the polypeptide.

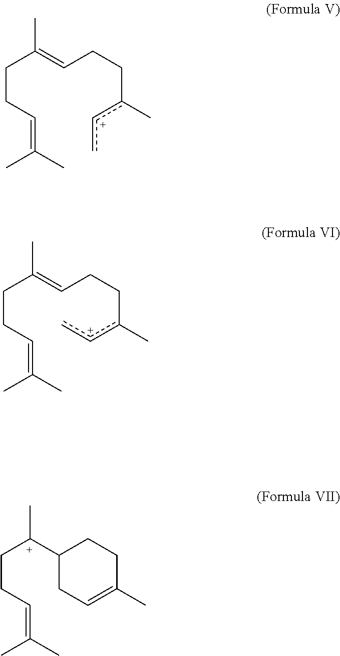

The term “sequence identity” or “sequence . . . identical to” as used herein defines a relationship between amino acid sequences or nucleic acid sequences and can be determined by comparing those sequences. Usually, sequence identities are determined by comparing two sequences over the whole length of the sequences but may also be compared only for a part of the sequences aligning with each other. Preferably, the sequence identities are compared over the whole length of the sequences, herein. Sequence identity refers to the degree of relatedness between polypeptide sequences or nucleic acid sequences. It will be expressed in the percentage of identical amino acids or nucleotides in two sequences compared to each other. Accordingly, upon aligning two sequences, the number of matching amino acids or nucleotides between those sequences is, in general, determined and put into relation to the total number of amino acids or nucleotides in the aligned sequence or sequence part. For instance, variant sequences may be defined by their sequence identity when compared to a parent sequence, i.e. an amino acid sequence as shown in any one of SEQ ID NO: 1 or a nucleic acid sequence as shown in SEQ ID NOs: 2, 3 or 5. To determine the percent-identity between two sequences in a first step a pairwise sequence alignment is generated between those two sequences, wherein the two sequences are aligned over their complete, entire or full length (i.e. a pairwise global alignment). The alignment is generated with a program or software described herein. The preferred alignment for the purpose of this invention is that alignment, from which the highest sequence identity can be determined.

Sequence alignments can be generated with a number of software tools, such as Needleman and Wunsch algorithm—Needleman, Saul B. & Wunsch, Christian D. (1970): “A general method applicable to the search for similarities in the amino acid sequence of two proteins”, Journal of Molecular Biology 48 (3): 443-453. This algorithm is, for example, implemented into the “NEEDLE” program, which performs a global alignment of two sequences. The NEEDLE program is contained within, for example, the European Molecular Biology Open Software Suite (EMBOSS). EMBOSS—a collection of various programs: The European Molecular Biology Open Software Suite (EMBOSS), Trends in Genetics 16 (6), 276 (2000).

BLOSUM (BLOcks SUbstitution Matrix)—typically generated on the basis of alignments of conserved regions, e.g., of protein domains (Henikoff S, Henikoff J G: “Amino acid substitution matrices from protein blocks”, Proceedings of the National Academy of Sciences of the USA; 1992 Nov. 15; 89(22): 10915-9). One out of the many BLOSUMs is “BLOSUM62”, which is often the “default” setting for many programs, when aligning protein sequences. “BLAST” (Basic Local Alignment Search Tool) consists of several individual programs (BlastP, BlastN), which are mainly used to search for similar sequence in large sequence databases.

BLAST programs also create local alignments. Typically used is the BLAST interface provided by NCBI (National Centre for Biotechnology Information), which is the improved version (“BLAST2”). The “original” BLAST: Altschul, S. F., Gish, W., Miller, W., Myers, E. W. & Lipman, D. J. (1990): “Basic local alignment search tool”, J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997): “Gapped BLAST and PSI-BLAST: a new generation of protein database search programs”, Nucleic Acids Res. 25:3389-3402.

“Sequence identity” as used herein is, preferably, the value as determined by the EMBOSS Pairwise Alignment Algorithm “Needle”. In particular, the NEEDLE program from the EMBOSS package can be used (version 2.8.0 or higher, EMBOSS: The European Molecular Biology Open Software Suite—Rice, P., et al., Trends in Genetics (2000) 16: 276-277; http://emboss.bioinformatics.nl) using the NOBRIEF option (‘Brief identity and similarity’ to NO) which calculates the “longest-identity”. The identity between the two aligned sequences is calculated in such a case as follows: Number of corresponding positions in the alignment showing an identical amino acid in both sequences divided by the total length of the alignment after subtraction of the total number of gaps in the alignment. For alignment of amino acid sequences, the default parameters are: Matrix=Blosum62; Open Gap Penalty=10.0; Gap Extension Penalty=0.5. For alignment of nucleic acid sequences, the default parameters are: Matrix=DNAfull; Open Gap Penalty=10.0; Gap Extension Penalty=0.5.

The term “zizaene” as used herein is defined as the compound shown above as Form. I (CAS: 18444-94-5) as well as all stereoisomeric forms thereof, in particular the enantiomeric form thereof, as well as all possible mixtures of said compound and its stereoisomeric forms. Preferably, however, “zizaene” is to be understood as the compound shown as Form. I only.

The term “zizanal” as used herein is defined as the compound shown above as Form. II (CAS: 82509-29-3) as well as all stereoisomeric forms thereof, in particular the enantiomeric form thereof, as well as all possible mixtures of said compound and its stereoisomeric forms. Preferably, however, “zizanal” is to be understood as the compound shown as Form. II only.

The term “khusimol” as used herein is defined as the compound shown above as Form. III (CAS: 16223-63-5) as well as all stereoisomeric forms thereof, in particular the enantiomeric form thereof, as well as all possible mixtures of said compound and its stereoisomeric forms. Preferably, however, “zizanal” is to be understood as the compound shown as Form. III only.

The term “khusimone” as used herein is defined as the compound shown above as Form. IV (CAS: 30557-76-7) as well as all stereoisomeric forms thereof, in particular the enantiomeric form thereof, as well as all possible mixtures of said compound and its stereoisomeric forms. Preferably, however, “khusimone” is to be understood as the compound shown as Form. IV only.

Herein, the term “unknown terpene X” is defined as the compound or mixture of compounds, preferably terpenes and/or terpenoids, exhibiting the mass spectrum depicted in FIG. 4, with peaks at least at m/z=of 91 (highest peak), 108, 117, 133 and 204, preferably at least at m/z=41, 55, 65, 77, 91 (highest peak), 105, 108, 117, 133, 145, 189 and 204, wherein “unknown” shall be understood in such a way, that the chemical structure has not yet been determined.

Herein, the term “unknown terpene Y” is defined as the compound or mixture of compounds, preferably terpenes and/or terpenoids, exhibiting the mass spectrum as depicted in FIG. 5, with peaks at least at m/z=91, 105, 133 (highest peak), 134 and 204, preferably at least at m/z=41, 55, 63, 70, 77, 83, 91, 105, 117, 133 (highest peak), 134, 141, 157 and 204, wherein “unknown” shall be understood in such a way, that the chemical structure has not yet been determined.

Herein, “% of total” means the area percentage of a given compound in an according gas chromatogram (GC) obtained by subjecting the zizaene-containing composition to GC-FID (gas chromatography using a flame ionization detector) and is preferably equivalent to the mol % in the zizaene-containing composition.

“CrZIS” as used herein means the zizaene synthase from Capsella rubella according to the present invention, whereas “VzZIS” refers to the known zizaene synthase from Vetiveria zizanoides.

Preferably, the polypeptide exhibiting zizaene synthase activity is or is based on a polypeptide from the taxonomical families Camelineae or Brassicaceae, more preferably from the genera Capsella, in particular C. rubella, Arabidopsis, in particular A. thaliana or A. lyrata, Brassica, in particular B. campestris, B. rapa, B. napus, B. oleracea or B. cretica, Eutrema, in particular E. salsugineum, Noccaea, in particular N. caerulescens, or Raphanus, in particular R. sativus, wherein the genera Capsella, Arabidopsis and Brassica are especially preferred.

Variant amino acid or nucleic acid sequences to the sequence of SEQ ID NO: 1 or the sequences of SEQ ID NOs: 2, 3 and 5, respectively, may be naturally occurring variations such as allelic variants or orthologous, paralogous or homologous variants. Alternatively, such sequences may be artificially generated, e.g. in an attempt to improve a property of the enzyme or nucleic acid (e.g. improved expression of the enzyme or increased enzymatic activity of the enzyme) by a biological technique known to the person skilled in the art, such as e.g. molecular evolution or rational design, or by using a mutagenesis technique known in the art or described elsewhere herein (random mutagenesis, site-directed mutagenesis, directed evolution, gene recombination, etc.). Typically, variants of the polypeptides with zizaene synthase activity according to the invention are polypeptides with one or several amino acid substitutions compared to the amino acid sequence of SEQ ID NO: 1, preferably, artificial amino acid sequences.

Variant nucleic acid sequences may differ from the sequences of SEQ ID NOs: 2, 3 and 5 due to at least one nucleotide substitution, addition and/or deletion. It will be understood that polynucleotides comprising such variant nucleic acid sequences as referred to herein, preferably, are capable of hybridizing to a nucleic acid sequence as shown in SEQ ID NOs: 2, 3 and 5 under stringent hybridization conditions.

Stringent hybridization conditions as referred to herein are, preferably, 6× sodium chloride/sodium citrate (SSC) at approximately 45° C., followed by one or more wash steps in 0.2×SSC, 0.1% SDS at 50 to 65° C. The skilled person knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1 to 5×SSC (pH 7.2). If organic solvent is present in the abovementioned buffer, for example 50% formamide, the temperature under standard conditions is approximately 42° C. The hybridization conditions for DNA:DNA hybrids are, preferably, 0.1×SSC and 20° C. to 45° C., preferably between 30° C. and 45° C. The above-mentioned hybridization temperatures are determined for example for a nucleic acid with approximately 100 bp (=base pairs) in length and a G+C content of 50% in the absence of formamide.

The skilled person knows how to determine the hybridization conditions required by referring to textbooks such as the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Thus, variant nucleic acid sequences can be derived which are capable of hybridizing under stringent hybridization conditions to a nucleic acid sequence as shown in SEQ ID NOs: 2, 3 or 5.

Typically, an amino acid sequence being a fragment of any one of the sequences listed in a) to d) of the method of the present invention consists of at least 20, at least 30, at least 40, at least 50, at least 100, at least 150 or at least 200 contiguous amino acids in length from the above-mentioned sequences or sequence variants, in order to exhibit zizaene synthase activity.

Preferably, an amino acid sequence or fragment thereof as listed in a) to e) of the inventive method of the present invention comprises at least one, preferably at least two, more preferably three Pfam domains. More preferably, the Pfam domains are selected from the group consisting of terpene synthase, N-terminal domain (PF01397.23), terpene synthase family, metal binding domain (PF03936.18) and terpene synthase family 2, C-terminal metal binding (PF19086.2). Pfam domains as referred to herein are to be analysed using version 35.0 of PFAM, for PFAM details see Pfam: The protein families database in 2021: J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman Nucleic Acids Research (2020) doi: 10.1093/nar/gkaa913, http://pfam.xfam.org/ or https://doi.org/10.1093/nar/gkaa913.

Preferably, inventive amino acid sequences or fragments thereof comprise at least 30, at least 50, at least 65 or all of the conserved residues shown in FIG. 7 by white letters (each one representing a specific amino acid) on black background. Conserved stretches of residues are indicated in FIG. 7 by a contiguous sequence of white letters (each one representing a specific amino acid) on black background. Preferably, conserved stretches consist of at least 2, at least 3 or at least 4 amino acids. Particularly preferred conserved stretches are those corresponding to the following amino acid positions of SEQ ID NO: 1: 119 to 123, 141 to 144, 159 to 161, 218 to 221, 223 to 226, 238 to 240, 253 to 255 and 440 to 442. The amino acid sequence or fragment thereof preferably comprises at least 4, more preferably at least 6 and most preferably all of these particularly preferred conserved stretches.

More preferably, inventive amino acid sequences or fragments thereof comprise at least 50, at least 70, at least 90, at least 105 or all of the conserved residues shown in FIG. 6 by white letters (each one representing a specific amino acid) on black background. Conserved stretches of residues are indicated in FIG. 6 by a contiguous sequence of white letters (each one representing a specific amino acid) on black background. Preferably, conserved stretches consist of at least 3, at least 5 or at least 7 amino acids. Particularly preferred conserved stretches are those corresponding to the following amino acid positions of SEQ ID NO: 1: 117 to 121, 139 to 143, 214 to 219, 242 to 246, 272 to 276, 286 to 293, 328 to 334, 336 to 340, 343 to 349, 371 to 380 and 414 to 418. The amino acid sequence or fragment thereof preferably comprises at least 5, more preferably at least 8 and most preferably all of these particularly preferred conserved stretches.

Preferably, the amino acid sequence or fragment thereof exhibits a SDVFX₁X₂F motif (cf. amino acid positions 129 to 135 of SEQ ID NO: 1 in FIG. 6), wherein S=serine, D=aspartic acid, V=valine, F=phenylalanine, X₁=any amino acid, preferably asparagine (N), lysine (K) or aspartic acid (D), and X₂=any amino acid, preferably, arginine (R), isoleucine (1) or lysine (K). This motif is surprisingly present in the known zizaene synthase from V. zizanoides (VzZIS) as well—even though the latter has a very low overall sequence identity compared to SEQ ID NO: 1.

The above-mentioned polypeptide exhibiting zizaene synthase activity may also be comprised in a fusion polypeptide. Such a fusion polypeptide comprises in addition to an amino acid sequence exhibiting zizaene synthase activity one or more additional amino acid sequences. Said additional amino acid sequences may be polypeptides having other enzymatic activities, such as farnesyl pyrophosphate synthases or cytochrome P450 monooxygenases as specified elsewhere herein or polypeptides or peptides having marker or label functions for, e.g., monitoring proper expression or for purification purposes, such as tags (MYC tag, FLAG tag, His tag, etc.) or fluorescent proteins (e.g. GFP, BFP, YFP or CFP).

The inventive method also refers to converting farnesyl pyrophosphate to zizaene by an enzyme comprising a first segment comprising a tag peptide and a second segment comprising a polypeptide exhibiting zizaene synthase activity. An enzyme comprising said first and said second segment may herein be referred to as a “tagged enzyme”.

The tag peptide is preferably selected from the group of nitrogen utilization proteins (NusA), thioredoxins (Trx), maltose-binding proteins (MBP), Glutathione S-transferases (GST), Small Ubiquitin-like Modifier (SUMO) or Calcium-binding proteins (Fh8), and functional homologues thereof. As used herein, a functional homologue of a tag peptide is a tag peptide having at least about the same effect on the solubility of the tagged enzyme, compared to the non-tagged enzyme. Typically, the homologue differs in that one or more amino acids have been inserted, substituted, deleted from, or extended to the peptide of which it is a homologue. The homologue may in particular comprise one or more substitutions of a hydrophilic amino acid for another hydrophilic amino acid, or of a hydrophobic amino acid for another. The homologue may, in particular, have a sequence identity of at least 40%, more in particular of at least 50%, preferably of at least 55%, more preferably of at least 60%, at least 70%, at least 75%, at least 80%, at least 85%, at least 90%, at least 95%, at least 98% or at least 99% sequence identity with the sequence of a NusA, Trx, MBP, GST, SUMO or Fh8.

Particularly suitable is the maltose-binding protein from Escherichia coli, or a functional homologue thereof. The use of a tagged enzyme according to the invention is in particular advantageous in that it may contribute to an increased production, especially increased cellular production of a terpenoid or a terpene, such as zizaene.

For improved solubility of the tagged enzyme (compared to the enzyme without the tag), the first segment of the enzyme is preferably bound at its C-terminus to the N-terminus of the second segment. Alternatively, the first segment of the tagged enzyme is bound at its N-terminus to the C-terminus of the second segment.

Further, the present invention is directed to a nucleic acid comprising a nucleotide sequence encoding a polypeptide comprising a first segment comprising a tag peptide, preferably a MBP, NusA, Trx, GST, SUMO or Fh8 tag or a functional homologue of any of these, and a second segment exhibiting zizaene synthase activity.

Further, the present invention is directed to a host cell comprising said nucleic acid encoding said tagged zizaene synthase. The host cell may in particular comprise a gene comprising any of these sequences or a functional homologue thereof.

The method according of the present invention may either consist of step i) to iii) or may comprise one or more additional steps. Such additional steps may be pre-treatment steps or steps required for obtaining the zizaene-containing composition such as purification steps, wherein, for example, the non-converted portion of farnesyl pyrophosphate and/or the at least one polypeptide exhibiting zizaene synthase activity may be removed from the zizaene-containing composition. The method may also comprise one or more additional steps, in which the zizaene and/or the at least one further compound provided in step iii) are further converted, e.g. oxidized.

The method may be carried out in vitro, e.g. in one or more reaction vials. Alternatively, the method may be carried out entirely or in part in a non-human host cell or transgenic organism as referred to herein below.

The above-mentioned conversion step may be carried out in vitro, i.e. in one or more suitable reaction vials containing all components required for the conversion. The skilled person is well-aware of how to adjust the reaction conditions such that the reaction will be carried out efficiently. For example, suitable buffers may be used to provide the components in an environment having a suitable pH and suitable salt concentrations. A suitable temperature in such a setting can be applied as well without further ado.

Alternatively, the conversion step may be carried out in a host cell as described herein below. It is to be understood that the host cell shall be capable of producing zizaene. If necessary, the host cell needs to be genetically modified in order to express the polypeptide exhibiting zizaene synthase activity. The host cell shall be cultivated under conditions and for a time sufficient to allow expression of said polypeptide and for conversion of farnesyl pyrophosphate into zizaene. Particularly preferred conditions are also described in the accompanying Examples below or known to those skilled in the art.

Yet, the conversion step of the method of the present invention may also be carried out in an organism, typically a multi-cellular organism such as a transgenic non-human organism as referred to herein below. Typically, said organism is genetically modified such that the polypeptide required for conversion of farnesyl pyrophosphate into zizaene is expressed. The skilled person is, however, well-aware of what conditions need to be applied depending on the choice of a given non-human transgenic organism.

In case the method of the invention is carried out in vivo, i.e. in a host cell or a non-human transgenic organism, it will be understood that said host cell or non-human transgenic organism shall express the polypeptide exhibiting zizaene synthase activity such that the conversion of farnesyl pyrophosphate into zizaene can be carried out in said host cell or non-human transgenic organism.

In the method of the present invention, the polypeptide exhibiting zizaene synthase activity is preferably encoded by a heterologous polynucleotide, which is preferably comprised by a vector or gene construct.

The term “heterologous polynucleotide” in this context means that the polynucleotide encoding the polypeptide exhibiting zizaene synthase activity is not naturally occurring in the host cell or organism into which it is introduced. Thus, a heterologous polynucleotide originates from a first species or is an artificially modified polynucleotide, while the host cell or non-human transgenic organism is from a second species that differs from said first species. A heterologous polynucleotide may be comprised in a vector or gene construct as specified herein below. Alternatively, it may be introduced into the genome of a host cell or non-human transgenic organism such that upon integration into the genome the polypeptide exhibiting zizaene synthase activity encoded by said heterologous polynucleotide will be expressed. Typically, the heterologous polynucleotide shall be integrated into the genome of the host cell or non-human transgenic organism at a locus that allows expression of the heterologous polynucleotide, e.g., in proximity to an endogenous promoter.

The term “vector” preferably encompasses phages, plasmids, cosmids, viral vectors as well as artificial chromosomes, such as bacterial or yeast artificial chromosomes (YAC). The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion.

Vectors can be introduced into prokaryotic or eukaryotic cells via conventional transformation or transfection techniques. The terms “transformation” and “transfection”, conjugation and transduction, as used in the present context, are intended to comprise a multiplicity of prior art processes for introducing foreign nucleic acids (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in Sambrook et al. (loc. cit.) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques. Should the vector be a virus, it may be packaged in vitro using an appropriate packaging cell line prior to application to host cells.

Preferably, the vector referred to herein is suitable as a cloning vector, i.e. replicable in microbial systems. Such vectors ensure efficient cloning in bacteria and, preferably, yeasts or fungi and make possible the stable transformation of plants. Those which must be mentioned are, in particular, various binary and co-integrated vector systems which are suitable for the T DNA-mediated transformation. Such vector systems are, as a rule, characterized in that they contain at least the vir genes, which are required for the Agrobacterium-mediated transformation, and the sequences which delimit the T-DNA (T-DNA border). These vector systems, preferably, also comprise further cis-regulatory regions such as promoters and terminators and/or selection markers with which suitable transformed host cells or organisms can be identified. While co-integrated vector systems have vir genes and T DNA sequences arranged on the same vector, binary systems are based on at least two vectors, one of which bears vir genes, but no T-DNA, while a second one bears T DNA, but no vir gene.

As a consequence, the last-mentioned vectors are relatively small, easy to manipulate and can be replicated both in E. coli and in Agrobacterium. These binary vectors include vectors from the pBIB-HYG, pPZP, pBecks, pGreen series. Preferably used in accordance with the invention are Bin19, pBl101, pBinAR, pGPTV and pCAMBIA. An overview of binary vectors and their use can be found in Hellens et al., Trends in Plant Science (2000) 5, 446-451. Furthermore, by using appropriate cloning vectors, the polynucleotides can be introduced into host cells or organisms such as plants or animals and, thus, be used in the transformation of plants, such as those which are published and cited in: Plant Molecular Biology and Biotechnology (CRC Press, Boca Raton, Florida), chapter 6/7, pp. 71-119 (1993); F. F. White, Vectors for Gene Transfer in Higher Plants, in: Transgenic Plants, vol. 1, Engineering and Utilization, Ed.: Kung and R. Wu, Academic Press, 1993, 15-38; B. Jenes et al., Techniques for Gene Transfer, in: Transgenic Plants, vol. 1, Engineering and Utilization, Ed.: Kung and R. Wu, Academic Press (1993), 128-143; Potrykus 1991, Annu. Rev. Plant Physiol. Plant Molec. Biol. 42, 205 225.

More preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX (Pharmacia Biotech Inc; Smith 1988, Gene 67:31-40), pMAL (New England Biolabs, Beverly, MA) and pRIT5 (Pharmacia, Piscataway, NJ), where glutathione S transferase (GST), maltose E-binding protein and protein A, respectively, are fused with the recombinant target protein.

Examples of suitable inducible non-fusion E. coli expression vectors are, inter alia, pTrc (Amann 1988, Gene 69:301-315) and pET 11d (Studier 1990, Methods in Enzymology 185, 60-89). The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident lambda-prophage which harbours a T7 gn1 gene under the transcriptional control of the lacUV 5 promoter. The skilled person is familiar with other vectors which are suitable in prokaryotic organisms; these vectors are, for example in E. coli pLG338, pACYC184, pACYCDuet-1 (Novagen, Merck, Germany), the pBR series such as pBR322, the pUC series such as pUC18 or pUC19, the M113mp series, pKC30, pRep4, pHS1, pHS2, pPLc236, pMBL24, pLG200, pUR290, pIN-111113-B1, lambdagt11 or pBdCI, in Streptomyces pIJ101, pIJ364, pIJ702 or pIJ361, in Bacillus pUB110, pC194 or pBD214, in Corynebacterium pSA77 or pAJ667.

Examples of vectors for expression in the yeast S. cerevisiae comprise pYep Sec1 (Baldari 1987, Embo J. 6:229-234), pMFa (Kurjan 1982, Cell 30:933-943), pJRY88 (Schultz 1987, Gene 54:113-123) and pYES2 (Invitrogen Corporation, San Diego, CA). Vectors and processes for the construction of vectors which are suitable for use in other fungi, such as the filamentous fungi, comprise those which are described in detail in: van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells (for example Sf9 cells) comprise the pAc series (Smith 1983, Mol. Cell Biol. 3:2156-2165) and the pVL series (Lucklow 1989, Virology 170:31-39).

Yet, the vector may be an integration vector. An integration vector refers to a DNA molecule, linear or circular, that can be incorporated, e.g. into the genome of a microorganism, such as a bacteria genome, and provides for stable inheritance of a gene encoding a polypeptide of interest, such as the zizaene synthase of the invention. The integration vector generally comprises one or more segments comprising a gene sequence encoding a polypeptide of interest under the control of (i.e. operably linked to) additional nucleic acid segments that provide for its transcription.

Such additional segments may include promoter and terminator sequences and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors.

For example, a DNA sequence for a signal peptide leader can be fused in-frame to a nucleic acid of the invention so that the zizaene synthase of the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino- or carboxyl-terminal end of the polypeptide.

The term “gene construct” as used herein refers to polynucleotides comprising the polynucleotide of the invention and additional functional nucleic acid sequences. A gene construct according to the present invention is, preferably, a linear DNA molecule. Typically, a gene construct in accordance with the present invention may be a targeting construct which allows for random or site-directed integration of the targeting construct into genomic DNA. Such target constructs, preferably, comprise DNA of sufficient length for either homologous or heterologous recombination as described in detail below. In both cases, the construct must be, preferably, impeccable, with structures to control gene expression, such as a promoter, a site of transcription initiation, a site of polyadenylation, and a site of transcription termination.

Preferably, the method of the present invention comprises the step of obtaining said zizaene-containing composition. The term “obtaining” as used herein refers to providing the zizaene-containing composition at any degree of purity. Thus, the method of the invention may encompass one or more purification steps. The purification techniques, which need to be applied, depend on how the method of the present invention has been carried out. For example, if the method has been carried out in vitro, i.e. in reaction vials using isolated components such as isolated enzymes, adducts and auxiliary components such as reaction buffers, it will be understood that less purification is required. However, if the method is carried out in vivo, i.e. in a non-human host cell or transgenic organism as defined herein, further purification and pre-treatment steps may be necessary. Typically, the host cells need to be harvested and the harvested cells will be lysed in order to release the zizaene-containing composition from said cells. Subsequent purification steps shall remove the cell debris as well as aiming at purifying the zizaene-containing composition from the remaining components.

Moreover, if the steps are carried out in vivo in animals or plants, even further pre-treatment and/or purification steps may be required. The skilled person is well-aware of suitable pre-treatment and/or purification steps depending on the given circumstances under which the method may be carried out. Purification techniques to be envisaged may be extraction techniques, chromatography, such as LC, GC or HPLC, size-exclusion chromatography, affinity chromatography, distillation, centrifugation, filtration and the like. Pre-treatment steps to be envisaged may be harvesting, heat treatment, ultra-sonic treatment, treatment with chemicals and/or enzymes, and the like. Particularly preferred measures are described in the accompanying Examples below.

The method of the present invention is preferably carried out in a non-human host cell.

The term “host cell” as used herein relates to a prokaryotic or eukaryotic cell, which is capable of converting farnesyl pyrophosphate into zizaene, wherein said conversion is carried out by a polypeptide exhibiting zizaene synthase activity. Thus, the host cell of the invention is capable of expressing the polypeptide exhibiting zizaene synthase activity. Preferably, said polypeptide exhibiting zizaene synthase activity may be encoded by the heterologous polynucleotide or vector or gene construct of the invention. The host cell is, typically transformed with said heterologous polynucleotide, vector or gene construct such that the polypeptide exhibiting zizaene synthase activity specified above can be expressed. The transformed vector or gene construct may be maintained as a non-integrated vector, for example, a plasmid, or alternatively, may be integrated into the host cell genome as specified herein in more detail.

In one aspect of the invention the host cell of the invention is a transgenic cell transgenic for the nucleic acid encoding the zizaene synthases of the invention, preferably a transgenic non-plant cell such as a transgenic microorganism cell.

A host cell according to the invention may be produced based on standard genetic and molecular biology techniques that are generally known in the art, e.g. as described in Sambrook, J., and Russell, D. W. “Molecular Cloning: A Laboratory Manual” 3d ed., Cold Spring Harbor Laboratory Press, Cold Spring Harbor, NY, (2001); and F. M. Ausubel et al., eds., “Current protocols in molecular biology”, John Wiley and Sons, Inc., New York (1987), and later supplements thereto.

Preferably, said host cell is a non-human host cell selected from the group consisting of a bacterial cell such as a cyanobacterial cell, a fungal cell such as a yeast cell, a plant cell such as an algal cell and a non-human animal cell such as a non-human mammalian cell. More preferably, the host cell can be selected from any one of the following organisms:

Bacteria: A bacterial host cell can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus, Lactococcus, Ensifer or Pantoea.

Gram-positive: Bacillus, Streptomyces: Useful gram-positive bacterial host cells include, but are not limited to, a Bacillus cell, e.g. Bacillus alkalophilus, Bacillus amyloliquefaciens, Bacillus brevis, Bacillus circulans, Bacillus clausii, Bacillus coagulans, Bacillus firmus, Bacillus jautus, Bacillus lentus, Bacillus licheniformis, Bacillus megaterium, Bacillus pumilus, Bacillus stearothermophilus, Bacillus subtilis and Bacillus thuringiensis. Most preferred, the prokaryote is a Bacillus cell, preferably a Bacillus cell of Bacillus subtilis, Bacillus pumilus, Bacillus licheniformis or Bacillus lentus.

Some other preferred bacteria include strains of the order Actinomycetales, preferably Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g. M. virescens.

Gram-negative: Escherichia, Pseudomonas, Rhodobacter, Paracoccus, Ensifer or Pantoea species: Preferred gram-negative bacteria are Escherichia coli, Pseudomonas sp., preferably Pseudomonas purrocinia (ATCC 15958) or Pseudomonas fluorescens (NRRL B-11) or Pseudomonas denitrificans, Rhodobacter capsulatus or Rhodobacter sphaeroides, Paracoccus carotinifaciens, Paracoccus zeaxanthinifaciens, Pantoea ananatis or Sinorhizobium meliloti also known as Ensifer meliloti.

Fungi: Aspergillus, Fusarium, Trichoderma: The host cell may be a fungal cell. “Fungi” as used herein includes the phyla Ascomycota, Basidiomycota, Chytridiomycota and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g. Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes. Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A). Further preferred fungi include strains belonging to the subdivision Zygomycotina, class Mycoraceae, e.g. Rhizopus or Mucor, in particular Mucor hiemalis.

Yeasts: Pichia, Saccharomyces: The fungal host cell may be a yeast cell. Yeast as used herein includes ascosporogenous yeast (Endomycetales), basidiosporogenous yeast and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g. genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium and Filobasidiella. Yeasts belonging to the Fungi Imperfecti are divided into two families, Sporobolomycetaceae (e.g. genera Sporobolomyces and Bullera) and Cryptococcaceae (e.g. genus Candida).

Besides fungi, non-human eukaryotic host cells further include, without limitation, a non-human animal cell, in particular a non-human mammal cell, an avian cell, a reptilian cell or an insect cell, or a plant cell.

Most preferably, the host cell is a bacterial host cell, in particular a Rhodobacter, preferably R. sphaeroides, host cell or an Escherichia, preferably E. coli, host cell, especially preferred a Rhodobacter, preferably R. sphaeroides, host cell.

Alternatively, but also preferably, the method of the present invention is carried out in a non-human transgenic organism.

The term “transgenic non-human organism” or “non-human transgenic organism” as used herein refers to an organism which has been genetically modified in order to comprise the polynucleotide, vector or gene construct of the present invention. Said genetic modification may be the result of any kind of homologous or heterologous recombination event, mutagenesis or gene editing process. Accordingly, the transgenic non-human organism shall differ from its non-transgenic counterpart in that it comprises the non-naturally occurring (i.e. heterologous) polynucleotide, vector or gene construct in its genome. Non-human organisms envisaged as transgenic non-human organisms in accordance with the present invention are, preferably, muti-cellular organisms. Moreover, the non-human organisms are, preferably, animals or plants. Preferred animals are mammals, in particular laboratory animals such as rodents, e.g. mice, rats, rabbits or the like, or farming animals such as sheep, goat, cows, horses or the like. Preferred plants are crop plants or vegetables, in particular selected from the group consisting of Arabidopsis spp., Nicotiana spp., Cichorum intybus, Lactuca sativa, Mentha spp., Artemisia annua, tuber forming plants, oil crops, e.g. Brassica spp. or Brassica napus, flowering plants (angiosperms) which produce fruits and trees.

Methods for the production of transgenic non-human organisms are well known in the art; see e.g. Lee-Yoon Low et al., Transgenic Plants: Gene constructs, vector and transformation method. 2018. DOI.10.5772/intechopen.79369; Pinkert, C. A. (ed.) 1994. Transgenic animal technology: A laboratory handbook. Academic Press, Inc., San Diedo, Calif.; Monastersky G. M. and Robl, J. M. (ed.) (1995) Strategies in Transgenic Animal Science. ASM Press. Washington D.C); Sambrook, loc. cit, Ausubel, loc. cit).

Preferably, said non-human transgenic organism is a microorganism, a plant or a non-human animal, preferably a non-vertebrate animal, to be sacrificed. Accordingly, in accordance with the latter, methods of treating animals are not encompassed by the methods of the present invention.

The present invention also relates to a zizaene-containing composition obtainable with the inventive method as described herein above.

The zizaene-containing composition preferably comprises, besides zizaene, at least one, preferably at least two further compounds selected from the group consisting of α-funebrene, unknown terpene X, β-funebrene, unknown terpene Y, prezizaene, cis-muurola-4(15),5-diene, R-acoradiene, α-neocallitropsene and acorenol, preferably consisting of α-funebrene, β-funebrene, unknown terpene Y and R-acoradiene, more preferably consisting of α-funebrene and R-acoradiene.

Preferably, in the zizaene-containing composition the content of zizaene is at least 15 mol %, preferably at least 25 mol %, the content of α-funebrene is at least 10 mol %, preferably at least 15 mol %, and the content of β-acoradiene is at least 10 mol %, preferably at least 15 mol %.

The zizaene-containing composition preferably comprises, besides zizaene, at least one further compound selected from the group consisting of unknown terpene X, unknown terpene Y, cis-muurola-4(15),5-diene and α-neocallitropsene and acorenol, preferably unknown terpene Y.

Preferably, in the zizaene-containing composition the total content of β-copaene and acora-3,9-diene is 0.5 mol % or less, preferably 0.3 mol % or less, more preferably 0.1 mol % or less.

The present invention is also directed to a method for producing at least one terpene and/or terpenoid, which comprises the following subsequent steps:
i) providing farnesyl pyrophosphate and at least one polypeptide under conditions suitable for the polypeptide to convert at least a portion of the farnesyl pyrophosphate into bisabolyl cations (cf. Form. VII above);
ii) converting, by means of the polypeptide, at least a portion of the farnesyl pyrophosphate into bisabolyl cations;
iii) converting, by means of the polypeptide, the bisabolyl cations into at least one terpene and/or terpenoid; and
iv) optionally purifying one or more of the at least one terpene and/or terpenoid from the mixture,
wherein the at least one polypeptide is capable of converting at least a portion of farnesyl pyrophosphate into bisabolyl cations and comprises or consists of an amino acid sequence selected from the group consisting of:
a) an amino acid sequence as shown in SEQ ID NO: 1;
b) an amino acid sequence which is at least 55% identical to the amino acid sequences as shown in SEQ ID NO: 1;
c) an amino acid sequence encoded by a nucleic acid sequence as shown in any one of SEQ ID NOs: 2, 3 or 5;
d) an amino acid sequence encoded by a nucleic acid sequence which is at least 55% identical to a nucleic acid sequence as shown in any one of SEQ ID NOs: 2, 3 or 5; and
e) an amino acid sequence being a fragment of any one of the sequences listed in a) to d), said fragment being capable of converting at least a portion of farnesyl pyrophosphate into bisabolyl cations.

At that, step ii) of producing bisabolyl cations from farnesyl pyrophosphate preferably includes the intermediate formation of farnesyl cations (cf. Form. V above) and/or of nerolidyl cations (cf. Form. VI above), preferably the intermediate formation of both.

Said method may also include a further step v) of transforming at least one terpene produced in steps i) to iv) to a terpenoid, preferably by an oxidation reaction. Non-limiting examples of such terpenoids producible by said method are zizanal (cf. Form. II above), khusimol (cf. Form. III above) and khusimone (cf. Form. IV above).

Moreover, the present invention refers to the use of the polypeptide exhibiting zizaene synthase activity as described herein above, the non-human host cell or transgenic organism as described herein above or the heterologous polynucleotide, vector or gene construct as described herein above for producing a zizaene-containing composition, preferably a zizaene-containing composition as described herein above. The zizaene-containing composition obtainable with the inventive method may be used in flavour or fragrance applications, in cosmetic uses, in pharmaceuticals, as insect repellent or insect attractant, in agriculture, e.g. for crop protection or animal raising, or in anti-microbial applications.

At that, it is possible to provide a kit for producing a zizaene-containing composition, which comprises as components: i) the polypeptide exhibiting zizaene synthase activity as described herein above, ii) the non-human host cell or transgenic organism as described herein above and/or iii) the heterologous polynucleotide, vector or gene construct as described herein above.

The term “kit” as used herein refers to a collection of components required for carrying out the method of the present invention for producing a zizaene-containing composition. The kit shall include any of the afore-mentioned components i) to iii) either as a single component or any combinations thereof. Typically, the components of the kit are provided in separate containers or within a single container. The container also typically comprises instructions for carrying out the method of the present invention for producing a zizaene-containing composition. Moreover, the kit may preferably comprise further components which are necessary for carrying out the method of the invention such as incubation reagents, cultivation media, washing solutions, solvents and/or reagents or means required for purification of the zizaene-containing composition.

Further on, the present invention relates to a method for producing a composition comprising at least one, preferably at least two, more preferably all oxidation products of zizaene selected from the group consisting of zizanal (cf. Form. II), khusimol (cf. Form. III) and khusimone (cf. Form. IV), in which:
a) a zizaene-containing composition is provided by conducting the inventive method as described herein above; and
b) the zizaene-containing composition provided in step a) is enzymatically and/or chemically, preferably enzymatically, oxidized, such that at least a portion of the contained zizaene is converted into zizanal, khusimol and/or khusimone.

Preferably, said method may further comprise the step of obtaining said composition comprising at least one oxidation product of zizaene, i.e. according pre-treatment and/or purification steps.

In step b) of said method, the oxidation of the contained zizaene is, preferably, carried out enzymatically. Suitable enzymes that are capable oxidizing zizaene and, thus, converting it into zizanal, khusimol and/or khusimone are well known in the art. Preferably, cytochrome P450 monooxygenases (CYPs) or laccases are used for oxidizing zizaene into zizanal, khusimol and/or khusimone, especially such that are described in the international patent application WO 2013/064411 A1 (Firmenich SA) or the U.S. Pat. No. 6,200,786 B1 (Givaudan S.A.), respectively. Yet, the oxidation may also be carried out chemically.

Step b) of said method may also be carried out in vivo or in vitro, typically, dependent on how step a) is carried out. Thus, if step a) is carried out in vivo, e.g. in a non-human host cell or transgenic organism as specified herein, it is preferably envisaged that step b) is carried out in vivo as well and, preferably, in the same host cell or transgenic organism. Typically, the non-human host cell or transgenic organism shall be capable of carrying out the oxidation of zizaene into zizanal, khusimol and/or khusimone as well. Preferably, said host cell or transgenic organism may, thus, express a CYP as specified above. To this end, a heterologous polynucleotide encoding said CYP or a vector or gene construct comprising such a polynucleotide may be present in the non-human host cell or transgenic organism. How such heterologous polynucleotides, vectors or gene constructs may be introduced into the said host cell or non-human transgenic organism is well known in the art and described elsewhere herein in detail.

Alternatively, step b) of said method for producing a composition comprising at least one oxidation product of zizaene is carried out by a chemical process rather than an enzymatic one. Preferably, the chemical oxidation of zizaene is performed as disclosed, e.g., in A. Denicourt-Nowicki et al., “Catalytic Oxidation Processes for the Upgrading of Terpenes”, Catalysts 2019, 9(11), 893.

Preferably, in said method, at least one, preferably at least two further compounds, besides zizaene, selected from the group consisting of α-funebrene, unknown terpene X, β-funebrene, unknown terpene Y, prezizaene, cis-muurola-4(15),5-diene, β-acoradiene, α-neocallitropsene and acorenol, preferably consisting of α-funebrene, β-funebrene, unknown terpene Y and β-acoradiene, more preferably consisting of α-funebrene and β-acoradiene are contained in the zizaene-containing composition provided in step a) and at least one, preferably at least two of said compounds are oxidized in step b).

Said method may also include, subsequent to step b), one or more purification steps like a distillation to remove other compounds and, if desired, the ratio of the compounds may be altered by distillation.

The present invention refers to an oxidized composition comprising at least one, preferably at least two, more preferably all oxidation products of zizaene selected from the group consisting of zizanal, khusimol and khusimone obtainable with said method as well.

The present invention also refers to a non-human host cell as described herein above expressing the polypeptide exhibiting zizaene synthase activity as described herein above from the heterologous polynucleotide, vector or gene construct as described herein above, wherein the non-human host cell is transgenic for the polynucleotide encoding said polypeptide. Preferably, said non-human host cell produces a zizaene-containing composition or a composition comprising at least one, preferably at least two, more preferably all oxidation products of zizaene selected from the group consisting of zizanal, khusimol and khusimone.

Further on, the present invention relates to a non-human transgenic organism as described herein above expressing the polypeptide exhibiting zizaene synthase activity as described herein above from the heterologous polynucleotide, vector or gene construct as described herein above, wherein the non-human transgenic organism is transgenic for the polynucleotide encoding said polypeptide. Preferably, said non-human transgenic organism produces a zizaene-containing composition or a composition comprising at least one, preferably at least two, more preferably all oxidation products of zizaene selected from the group consisting of zizanal, khusimol and khusimone.

The non-human host cell, non-human transgenic organism and methods of the invention may comprise the polypeptide exhibiting zizaene synthase activity of the invention and in addition one or more further polypeptides exhibiting zizaene synthase activity, including the known zizaene synthase protein from V. zizanoides (VzZIS).

Last not least, the present invention refers to a polypeptide exhibiting zizaene synthase activity and comprising an amino acid sequence which is less than 100%, but at least 85%, preferably at least 90%, more preferably at least 95%, at least 96%, at least 97%, at least 98% and most preferably at least 99% identical to the amino acid sequence as shown in SEQ ID NO: 1 or a fragment thereof exhibiting zizaene synthase activity. Preferably, the polypeptide comprises the conserved stretches and/or the SDVFX₁X₂F motif as defined herein above within the description of the inventive method.

Further preferred features and embodiments of said inventive polypeptide are described herein above within the description of the inventive method as well.

All references cited throughout this specification are herewith incorporated by reference in their entireties or with respect to the specifically mentioned disclosure content.

### DESCRIPTION OF FIGURES

FIG. 1: Schematic drawing of vector p-mev-SPppa-MBP-CrZIS for cloning and expressing CrZIS (=C. rubella zizaene synthase) in R. sphaeroides; mob=mobilization sequence, kan=kanamycin resistance gene, mvaA=3-hydroxy-3-methylglutaryl-coenzyme A reductase, idi=Isopentenyl-diphosphate Delta-isomerase, hcs=3-hydroxy-3-methylglutaryl-coenzyme A synthase, mvk=mevalonate kinase, pmk=phosphomevalonate kinase, mvd=Mevalonate Diphosphate Decarboxylase, MBP=maltose binding protein, rep=Origin of replication

FIG. 2: Schematic drawing of vector p-mev-SPppa-MBP-VzZIS for cloning and expressing VzZIS (=V. zizanoides zizaene synthase) in R. sphaeroides; cf. FIG. 1 for abbreviations

FIG. 3: Gas chromatogram (GC) of zizaene-containing compositions obtained with R. sphaeroides expressing CrZIS or VzVIS, respectively

FIG. 4: Mass spectrum (MS) of unknown terpene X obtained with R. sphaeroides expressing CrZIS-GC retention time (RT)=16.54 min

FIG. 5: MS of unknown terpene Y obtained with R. sphaeroides expressing CrZIS—RT=17.16 min

FIG. 6: Amino acid alignment of CrZIS having SEQ ID NO: 1 and proteins from different species. The alignment was made by using MUSCLE (MUltiple Sequence Comparison by Log-Expectation) and the standard parameters—numbering taken from SEQ ID NO: 1.

FIG. 7: Same as FIG. 6, but with alignment of three additional proteins having lower sequence identity compared to SEQ ID NO: 1.

The following sequences are referred to throughout the specification and in the accompanying sequence protocol:
SEQ ID NO: 1: Capsella rubella zizaene synthase (CrZIS) protein
SEQ ID NO: 2: nucleotide sequence encoding CrZIS protein in C. rubella
SEQ ID NO: 3: synthetic coding sequence for expression of CrZIS in E. coli
SEQ ID NO: 4: Vetiveria zizanoides zizaene synthase (VzZIS) protein
SEQ ID NO: 5: synthetic coding sequence for expression of CrZIS in R. sphaeroides
SEQ ID NO: 6: synthetic coding sequence for expression of VzZIS in R. sphaeroides
SEQ ID NO: 7: synthetic amino acid sequence 95% identical to SEQ ID NO: 1, variant no. 1
SEQ ID NO: 8: synthetic amino acid sequence 95% identical to SEQ ID NO: 1, variant no. 2
SEQ ID NO: 9: synthetic amino acid sequence 90% identical to SEQ ID NO: 1, variant no. 1
SEQ ID NO: 10: synthetic amino acid sequence 90% identical to SEQ ID NO: 1, variant no. 2
SEQ ID NO: 11: synthetic amino acid sequence 85% identical to SEQ ID NO: 1, variant no. 1
SEQ ID NO: 12: synthetic amino acid sequence 85% identical to SEQ ID NO: 1, variant no. 2
SEQ ID NO: 13: ROG872 Uncharacterized protein Capsella rubella (83% identical to SEQ ID NO: 1)
SEQ ID NO: 14: R0F0Z3 Uncharacterized protein (Fragment) Capsella rubella (83% identical to SEQ ID NO: 1)
SEQ ID NO: 15: Q4KSH9 Alpha-barbatene synthase Arabidopsis thaliana (82% identical to SEQ ID NO: 1)
SEQ ID NO: 16: A0A178UD70 Uncharacterized protein Arabidopsis thaliana (82% identical to SEQ ID NO: 1)
SEQ ID NO: 17: D7MLJ7 Terpene synthase/cyclase family protein Arabidopsis lyrata subsp. lyrata (81% identical to SEQ ID NO: 1)
SEQ ID NO: 18: A0A5S9YBB7 Uncharacterized protein Arabidopsis thaliana (81% identical to SEQ ID NO: 1)
SEQ ID NO: 19: synthetic amino acid sequence 80% identical to SEQ ID NO: 1, variant no. 1
SEQ ID NO: 20: synthetic amino acid sequence 80% identical to SEQ ID NO: 1, variant no. 2
SEQ ID NO: 21: synthetic amino acid sequence 75% identical to SEQ ID NO: 1
SEQ ID NO: 22: M4CD84 Uncharacterized protein Brassica rapa subsp. pekinensis (70% identical to SEQ ID NO: 1)
SEQ ID NO: 23: V4LIE4 Uncharacterized protein Eutrema salsugineum (70% identical to SEQ ID NO: 1)
SEQ ID NO: 24: A0A397XL88 Uncharacterized protein Brassica campestris (70% identical to SEQ ID NO: 1)
SEQ ID NO: 25: A0A1J3JN53 Alpha-barbatene synthase Noccaea caerulescens (65% identical to SEQ ID NO: 1)
SEQ ID NO: 26: A0A5S9XTN4 Uncharacterized protein Arabidopsis thaliana (55% identical to SEQ ID NO: 1)
SEQ ID NO: 27: Q9T0K1 (Z)-gamma-bisabolene synthase 2 Arabidopsis thaliana (55% identical to SEQ ID NO: 1)
SEQ ID NO: 28: Capsella bursa-pastoris zizaene synthase (CbpZIS) protein (95% identical to SEQ ID NO: 1)
SEQ ID NO: 29: synthetic coding sequence for expression of CbpZIS in R. sphaeroides Examples

The Examples shall merely illustrate the invention. They shall not, whatsoever, be construed as limiting the scope.

### Example 1: Identifying a Zizaene Synthase from C. rubella

An amino acid sequence, which is shown in the UniProt database under the accession no. R0GUH0 (cf. SEQ ID NO: 1) was extracted from the published Capsella rubella genome (https://www.uniprot.org/uniprotkb/R0GUH0/entry).

C. rubella genome sequencing citation: Slotte, T., Hazzouri, K., Agren, J. et al., “The Capsella rubella genome and the genomic consequences of rapid mating system evolution”, Nature Genetics 45, 831-835 (2013), https://doi.org/10.1038/ng.2669.

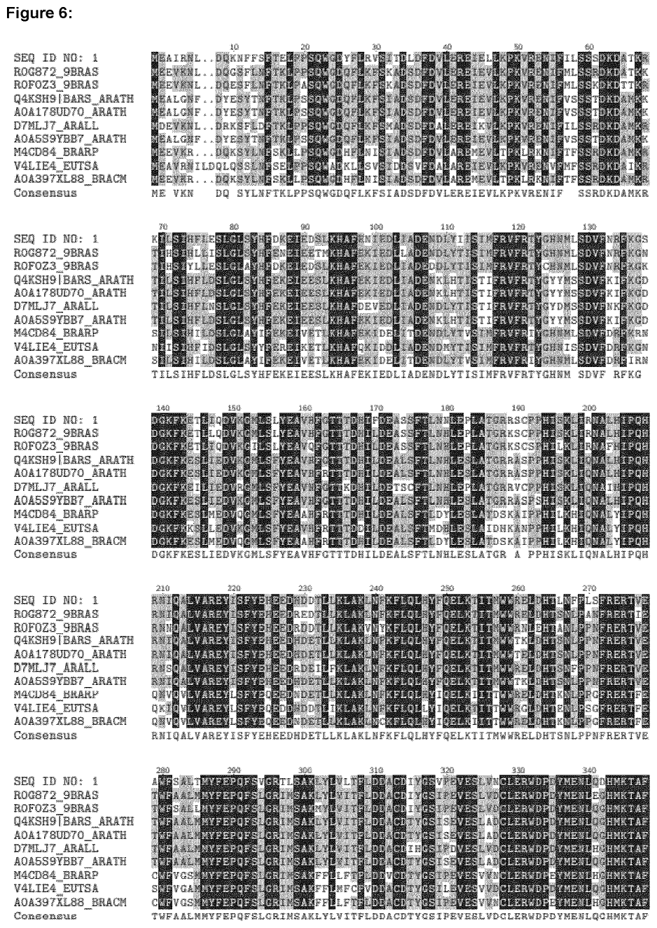

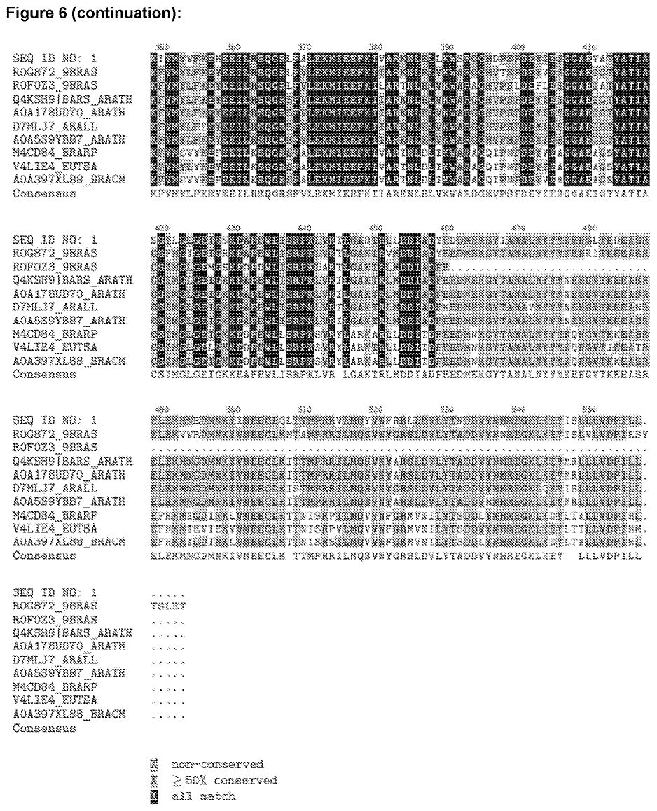

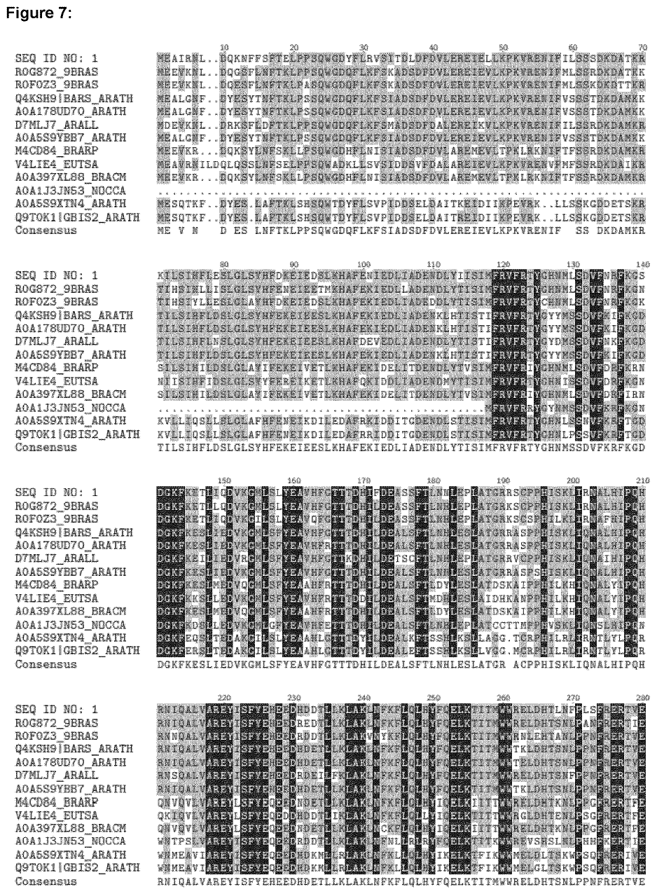

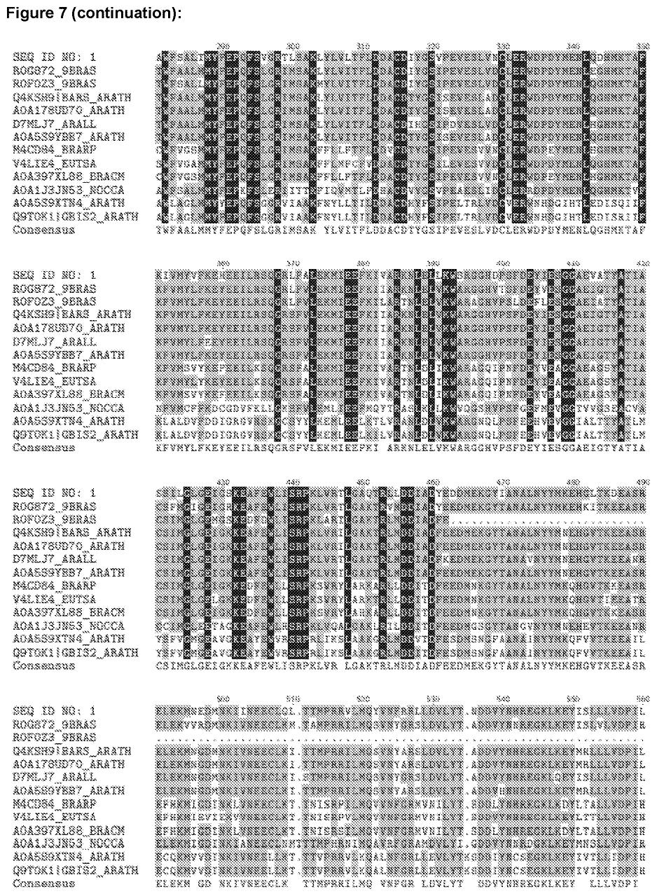

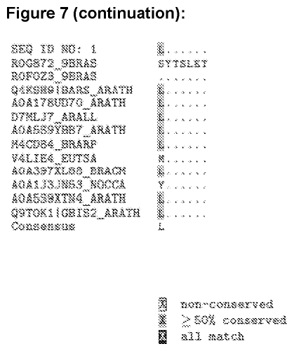

In a BLASTP analysis (nr database), the best blast hits (83% identical) are with two sequences which are each annotated as an uncharacterized protein from C. rubella. Four other sequences map within 80% identity, these sequences are found in the genus Arabidopsis. A MUSCLE alignment of SEQ ID NO: 1 and related sequences is shown in FIG. 6 and FIG. 7.

Sequence identity to the known zizaene synthase protein from Vetiveria zizanoides (GenBank accession no. AJQ30127) is only 29%. C. rubella is a plant very unrelated to V. zizanoides and not known to contain zizaene or zizaene-related metabolites.

Said amino acid sequence is encoded in C. rubella by a nucleic acid sequence (RefSeq no. XP_006281945.1; cf. SEQ ID NO: 2) annotated as alpha-barbatene synthase, based on homology to a barbatene synthase from Arabidopsis thaliana (GenBank accession no. AY876386).

### Example 2: Cloning for Expression in E. coli

To test whether the above amino acid sequence SEQ ID NO: 1 has zizaene synthase activity, it was expressed in Escherichia coli. For this, a nucleic acid sequence encoding R0GUH0 for E coli expression and having according linker sequences (cf. SEQ ID NO: 3) was synthesized using a standard sequence service provider (Genscript Biotech Corp., US) and cloned in expression vector pACYCDuet-1 (Novagen, Merck, Germany) using BamHI and NotI restriction sites. The resulting plasmid was labelled pAC-CrZIS.

The plasmid was introduced in E. coli strain BL21 DE3 harbouring pMEV, which has been described in Schmidt et al. 2017 (Scientific Reports | 7: 862 | DOI:10.1038/s41598-017-00893-3). Transformants were selected on LB-agar plates+chloramphenicol (30 μg/ml)+kanamycin (30 μg/ml)+1% glucose. A positive transformant (tested by miniprep and restriction digestion) was labelled E. coli BL21-DE3-pMEV-pAC-CrZIS. Using the same method, a control strain harbouring pMEV and pACYCDuet-1 was created, and labelled BL21-DE3-pMEV-pACYC-DUET-1.

### Example 3: Production of Zizaene in E. coli

E. coli BL21-DE3-pMEV-pAC-CrZIS and BL21-DE3-pMEV-pACYC-DUET-1 were each inoculated in 5 ml LB liquid medium+chloramphenicol (30 μg/ml)+kanamycin (30 μg/ml)+1% glucose and incubated overnight at 37° C. and 250 rpm. Next day, the cultures were diluted 1:25 in 10 ml 2×YT medium+chloramphenicol (30 μg/ml)+kanamycin (30 μg/ml) and grown at 37° C. and 250 rpm until A600 was 0.5. Subsequently, 1 mM IPTG was added as inducer, 1 ml of n-dodecane was added to capture the products and the cultures were further incubated for 24 hours at 28° C. 250 rpm.

For GC-MS analysis the n-dodecane was separated from the cultures by centrifugation and diluted 200 times with ethyl acetate. 2 μL were analysed by GC-MS using a gas chromatograph (GC) as described in detail by Cankar et al. (FEBS Letters 588 (2014) 1001-1007).

Surprisingly, when compared to BL21-DE3-pMEV-pACYC-DUET-1, the BL21-DE3-pMEV-pAC-CrZIS strain produced zizaene.

### Example 4: Constructs for Expression in R. sphaeroides

The zizaene synthase from C. rubella (cf. SEQ ID NO: 1; in the following “CrZIS”) as well as the zizaene synthase from V. zizanoides (GenBank accession no. AJQ30127; cf. SEQ ID NO: 4; in the following “VzZIS”) were expressed in Rhodobacter sphaeroides.

For this, synthetic, codon-optimized nucleic acid sequences for expressing CrZIS or VzZIS, respectively, in Rhodobacter sphaeroides (cf. SEQ ID NO: 5 or SEQ ID NO: 6, respectively) were ordered from a standard sequence service provider (Genscript Biotech Corp., US).

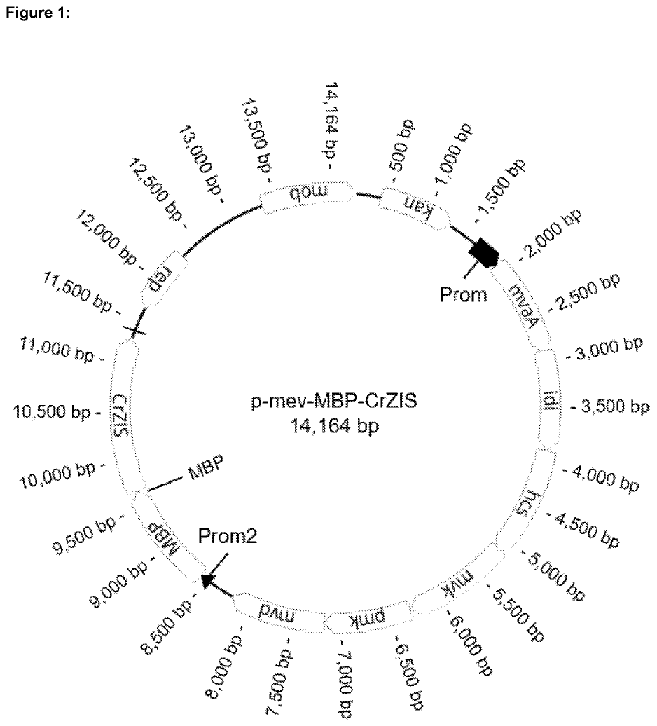

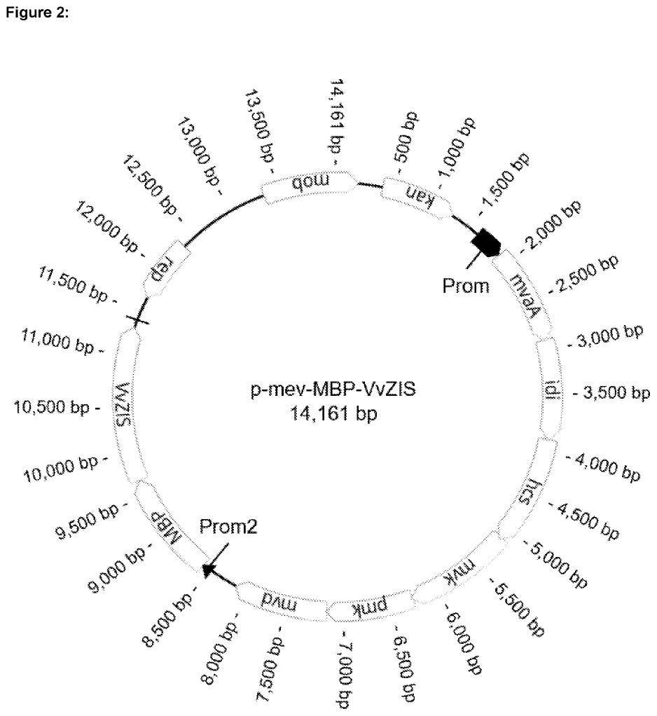

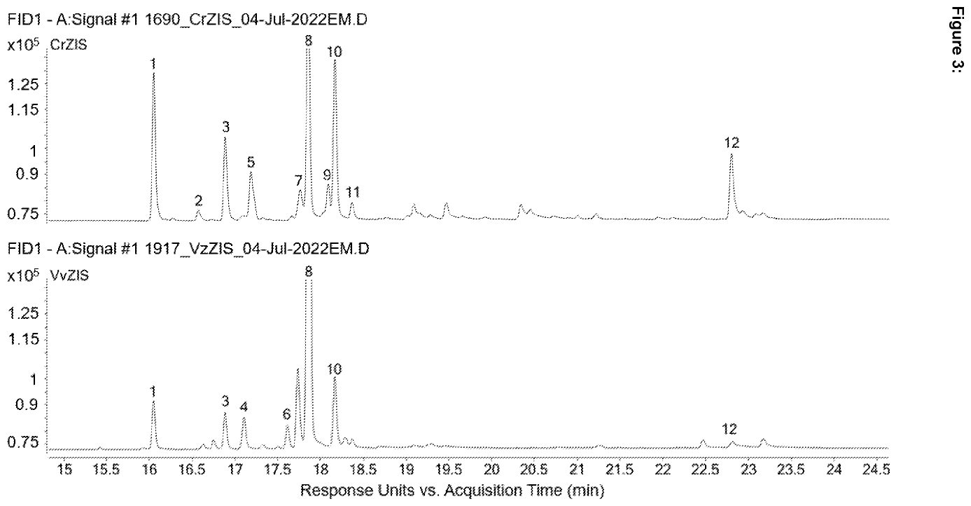

Each sequence was cloned in plasmid SPppa-MBP, as described in WO 2019/045568 A1 (Isobionics B.V.), and a mevalonate pathway was included, as described in WO 2019/045568 A1 as well, using BamHI and HindIII restriction sites. The resulting ligation mixture was transformed into E. coli S17-1 cells. Transfer of p-mev-SPppa-MBP-CrZIS (cf. FIG. 1) or p-mev-SPppa-MBP-VzZIS (cf. FIG. 2), respectively, from E. coli S17-1 to R. sphaeroides strain Rs265-9c by conjugation was performed using standard procedures (cf. U.S. Pat. No. 9,260,709 B2, Isobionics B.V.).

### Example 5: Production of Zizaene in R. sphaeroides

Seed cultures of Rs265-9c/p-mev-SPppa-MBP-CrZIS or Rs265-9c/p-mev-SPppa-MBP-VzZIS, respectively, were performed in 100 ml shake flasks without baffles with 20 ml RS102 medium with 100 mg/L neomycin and a loop of glycerol stock. Seed culture flasks were grown for 72 hours at 30° C. in a shaking incubator with an orbit of 50 mm at 110 rpm.

Shake flask production experiments were performed in 300 ml shake flasks with 2 bottom baffles. 20 ml of RS102 medium (as described in US 2020/0010822 A1, Isobionics B.V.) and neomycin to a final concentration of 100 mg/L were added to the flask together with 2 ml of sterile n-dodecane. The volume of the inoculum was adjusted to obtain a final OD600 value of 0.05 in 20 ml medium. The flasks were kept for 72 hours at 30° C. in a shaking incubator with an orbit of 50 mm at 110 rpm. Shake flask experiments were performed in duplicates.

For GC-MS analysis then, n-dodecane was separated from the culture by centrifugation and diluted 10 times with acetone.

Gas chromatography (GC) was performed on a Shimadzu GC2010 Plus equipped with a Restek RTX-5Sil MS capillary column (30 m×0.25 mm, 0.5 pm). The injector and FID detector temperatures were set to 280° C. and 300° C., respectively. Gas flow through the column was set at 40 ml/min. The oven initial temperature was 160° C., increased to 180° C. at a rate of 2° C./min, further increased to 300° C. at a rate of 50° C./min, and held for 3 min at that temperature. Injected sample volume was 1 μL with a 1:50 split ratio, and the nitrogen makeup flow was 30 ml/min.

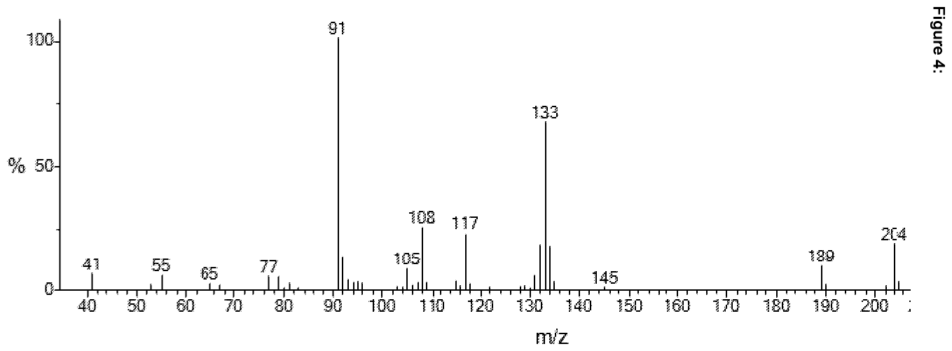

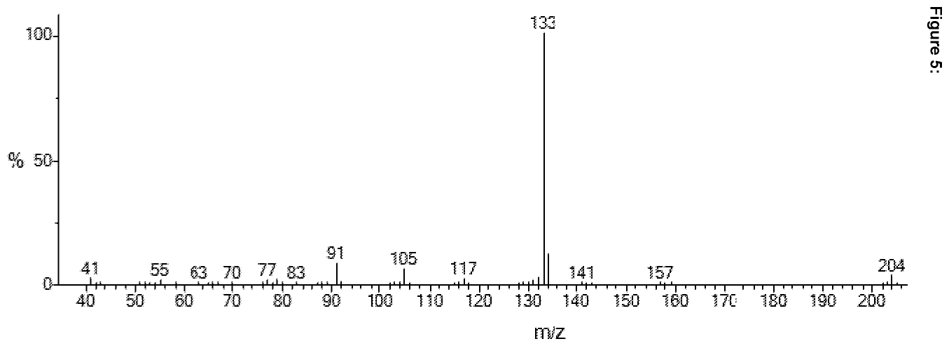

Compounds found in the n-dodecane layer from the Rs265-9c/p-mev-SPppa-MBP-CrZIS or Rs265-9c/p-mev-SPppa-MBP-VzZIS culture, respectively, were identified according to their retention time (RT) and quantified by integrating their peak area in the gas chromatogram (cf. FIG. 3). Analysis of peak areas revealed that both strains produce zizaene as the main product (cf. Tab. 1), as confirmed by mass spectrum.

Each strain produces side products as well, from which 4—α-funebrene, β-funebrene, prezizaene and β-acoradiene—are made by both, and several others are unique for one of the strains. In particular Rs265-9c/p-mev-SPppa-MBP-CrZIS (see column “CrZIS” in Tab. 1) produces significant amounts of an unknown terpene Y with retention index (RI)=1434 (for mass spectrum cf. FIG. 5) as well as a minor amount of another unknown terpene X with RI=1409 (for mass spectrum cf. FIG. 4), which are not produced by Rs265-9c/p-mev-SPppa-MBP-VzZIS (see column “VzZIS” in Tab. 1).

The presence of these new, hitherto unknown terpenes—in particular of the unknown terpene Y—in the zizaene-containing composition obtained with CrZIS results in a different organoleptic profile compared to the composition obtained with VzZIS.

### Example 6: Production of Zizaene in R. sphaeroides by Capsella bursa-pastoris Zizaene Synthase

An amino acid sequence (cf. SEQ ID NO: 28) was retrieved from the Capsella bursa-pastoris genome sequence (DOI: 10.1111/tpj.13563) by a TBLASTN search (https://blast.ncbi.nlm.nih.gov/Blast.cgi?PROGRAM=tblastn&PAGE_TYPE=BlastSearch&LINK_LOC=blasthome). C. bursa-pastoris genome sequencing citation: Kasianov et al., Plant J 2017 July; 91(2):278-291.

As determined in a BLASTP analysis (nr database), the sequence identity to the Capsella rubella zizaene synthase (cf. SEQ ID NO: 1) was 95%.

The zizaene synthase from C. bursa-pastoris (cf. SEQ ID NO: 28; in the following “CbpZIS”) as well as the zizaene synthase from V. zizanoides (GenBank accession no. AJQ30127; cf. SEQ ID NO: 4; in the following “VzZIS”) were expressed in Rhodobacter sphaeroides.

For this, synthetic, codon-optimized nucleic acid sequences for expressing CbpZIS or VzZIS, respectively, in Rhodobacter sphaeroides (cf. SEQ ID NO: 29 or SEQ ID NO: 6, respectively) were ordered from a standard sequence service provider (Genscript Biotech Corp., US).

Each sequence was cloned in plasmid SPppa-MBP, as described in WO 2019/045568 A1 (Isobionics B.V.), and a mevalonate pathway was included, as described in WO 2019/045568 A1 as well, using BamHI and HindIII restriction sites. The resulting ligation mixture was transformed into E. coli S17-1 cells. Transfer of p-mev-SPppa-MBP-CbpZIS or p-mev-SPppa-MBP-VzZIS, respectively, from E. coli S17-1 to R. sphaeroides strain Rs265-9c by conjugation was performed using standard procedures (cf. U.S. Pat. No. 9,260,709 B2, Isobionics B.V.).

Seed cultures of Rs265-9c/p-mev-SPppa-MBP-CbpZIS or Rs265-9c/p-mev-SPppa-MBP-VzZIS, respectively, according shake flask production experiments and GC-MS analysis were performed as in Example 5 above.

Compounds found in the n-dodecane layer from the Rs265-9c/p-mev-SPppa-MBP-CbpZIS or Rs265-9c/p-mev-SPppa-MBP-VzZIS culture, respectively, were identified according to their retention time (RT) and quantified by integrating their peak area in the gas chromatogram (data not shown). Analysis of peak areas revealed that both strains produce zizaene as the main product, as confirmed by mass spectrum (data not shown).

TABLE 1
Analysis of zizaene-containing compositions by GC-MS

| Peak | RT | | | | Lit. | % of total** | |
|---|---|---|---|---|---|---|---|
| # | (min) | RI | Compound | CAS no. | RI* | CrZIS | VzZIS |
| 1 | 16.03 | 1389 | α-funebrene | 50894- | 1385 | 18.9 | 2.7 |
| | | | | 66-1 | | | |
| 2 | 16.54 | 1409 | unknown terpene X | — | — | 1.4 | 0.0 |
| 3 | 16.86 | 1422 | β-funebrene | 79120- | 1418 | 8.4 | 1.6 |
| | | | | 98-2 | | | |
| 4 | 17.09 | 1431 | β-copaene | 18252- | 1430 | 0.0 | 1.9 |
| | | | | 44-3 | | | |
| 5 | 17.16 | 1434 | unknown terpene Y | — | — | 8.3 | 0.0 |
| 6 | 17.59 | 1452 | acora-3,9-diene | 55781- | 1442 | 0.0 | 1.0 |
| | | | | 50-5 | | | |
| 7 | 17.72 | 1457 | prezizaene | 31145- | 1452 | 1.7 | 5.3 |
| | | | | 21-8 | | | |
| 8 | 17.83 | 1461 | zizaene | 18444- | 1456 | 29.2 | 79.6 |
| | | | | 94-5 | | | |
| 9 | 18.06 | 1471 | cis-muurola- | 157477- | — | 2.4 | 0.0 |
| | | | 4(15),5-diene | 72-0 | | | |
| 10 | 18.14 | 1474 | β-acoradiene | 28477- | 1465 | 19.7 | 4.8 |
| | | | | 64-7 | | | |
| 11 | 18.34 | 1482 | α-neocallitropsene | 729602- | 1474 | 1.3 | 0.0 |
| | | | | 94-2 | | | |
| 12 | 22.78 | 1674 | acorenol | 28296- | 1667 | 2.8 | 0.1 |
| | | | | 85-7 | | | |

RT = retention time,
(Lit.) RI = (literature) retention index,
*from NIST database vs. 7,
**area percentage in GC-FID

1.-15. (canceled)

16. A method for producing at least one terpene and/or terpenoid comprising the following steps:
i) providing farnesyl pyrophosphate and at least one polypeptide under conditions suitable for the polypeptide to convert at least a portion of the farnesyl pyrophosphate into bisabolyl cations;
ii) converting, by means of the polypeptide, at least a portion of the farnesyl pyrophosphate into bisabolyl cations;
iii) converting, by means of the polypeptide, the bisabolyl cations into at least one terpene and/or terpenoid; and
iv) optionally purifying one or more of the at least one terpene and/or terpenoid from the mixture,
wherein the at least one polypeptide is capable of converting at least a portion of farnesyl pyrophosphate into bisabolyl cations and comprises or consists of an amino acid sequence selected from the group consisting of:
a) an amino acid sequence as shown in SEQ ID NO: 1;
b) an amino acid sequence which is at least 55% identical to the amino acid sequences as shown in SEQ ID NO: 1;
c) an amino acid sequence encoded by a nucleic acid sequence as shown in any one of SEQ ID NOs: 2, 3 or 5;
d) an amino acid sequence encoded by a nucleic acid sequence which is at least 55% identical to a nucleic acid sequence as shown in any one of SEQ ID NOs: 2, 3 or 5; and
e) an amino acid sequence being a fragment of any one of the sequences listed in a) to d), said fragment being capable of converting at least a portion of farnesyl pyrophosphate into bisabolyl cations.

17. A method for producing a zizaene-containing composition comprising the following steps:
i) providing farnesyl pyrophosphate and at least one polypeptide exhibiting zizaene synthase activity under conditions suitable for the polypeptide to convert at least a portion of the farnesyl pyrophosphate into zizaene;
ii) converting, by means of the polypeptide, at least a portion of the farnesyl pyrophosphate into bisabolyl cations; and
iii) producing a zizaene-containing composition by converting, by means of the polypeptide, the bisabolyl cations into zizaene and, optionally, at least one further compound,
wherein the at least one polypeptide comprises an amino acid sequence selected from the group consisting of:
a) an amino acid sequence as shown in SEQ ID NO: 1;
b) an amino acid sequence which is at least 55% identical to the amino acid sequences as shown in SEQ ID NO: 1;
c) an amino acid sequence encoded by a nucleic acid sequence as shown in any one of SEQ ID NOs: 2, 3 or 5;
d) an amino acid sequence encoded by a nucleic acid sequence which is at least 55% identical to a nucleic acid sequence as shown in any one of SEQ ID NOs: 2, 3 or 5; and
e) an amino acid sequence being a fragment of any one of the sequences listed in a) to d), said fragment exhibiting zizaene synthase activity.

18. The method of claim 16 wherein the zizaene-containing composition or the at least one terpene and/or terpenoid, respectively, produced in step iii) comprises at least one, preferably at least two further compounds selected from the group consisting of α-funebrene, unknown terpene X, β-funebrene, unknown terpene Y, prezizaene, cis-muurola-4(15),5-diene, β-acoradiene, α-neocallitropsene and acorenol, preferably consisting of α-funebrene, β-funebrene, unknown terpene Y and β-acoradiene, more preferably consisting of α-funebrene and β-acoradiene.

19. The method of claim 16 wherein in the zizaene-containing composition or the at least one terpene and/or terpenoid, respectively, produced in step iii) the content of zizaene is at least 15 mol %, preferably at least 25 mol %, the content of α-funebrene is at least 10 mol %, preferably at least 15 mol %, and the content of β-acoradiene is at least 10 mol %, preferably at least 15 mol %.

20. The method of claim 16 wherein the zizaene-containing composition or the at least one terpene and/or terpenoid, respectively, produced in step iii) comprises, at least one further compound selected from the group consisting of unknown terpene X, unknown terpene Y, cis-muurola-4(15),5-diene and α-neocallitropsene and acorenol, preferably unknown terpene Y.

21. The method of claim 16 wherein the zizaene-containing composition or the at least one terpene and/or terpenoid, respectively, produced in step iii) the total content of β-copaene and acora-3,9-diene is 0.5 mol % or less, preferably 0.3 mol % or less, more preferably 0.1 mol % or less.

22. The method of claim 16 wherein the method is carried out in a non-human host cell, preferably being selected from the group consisting of a bacterial cell such as a cyanobacterial cell, in particular an E. coli or R. sphaeroides cell, a fungal cell such as a yeast cell, a plant cell such as an algal cell and a non-human animal cell such as a non-human mammalian cell, or a non-human transgenic organism, preferably being a non-vertebrate animal to be sacrificed, a plant or a microorganism.

23. The method of claim 16 wherein the polypeptide is encoded by a heterologous polynucleotide, which is preferably comprised by a vector or gene construct.

24. A zizaene-containing composition obtainable with the method of claim 17.

25. Use of the polypeptide of claim 17 for producing the zizaene-containing composition.

26. Use of the host cell or transgenic organism as characterized in claim 22 for producing the zizaene-containing composition.

27. Use of the heterologous polynucleotide, vector or gene construct as characterized in claim 23 for producing the zizaene-containing composition.

28. A method for producing a composition comprising at least one, preferably at least two, more preferably all oxidation products of zizaene selected from the group consisting of zizanal, khusimol and khusimone comprising:
a) a zizaene-containing composition is provided by conducting the method of claim 17; and,
b) the zizaene-containing composition provided in step a) is enzymatically and/or chemically, preferably enzymatically, oxidized, such that at least a portion of the contained zizaene is converted into zizanal, khusimol and/or khusimone.

29. The method of claim 26 further comprising, besides zizaene, at least one, preferably at least two further compounds selected from the group consisting of α-funebrene, unknown terpene X, β-funebrene, unknown terpene Y, prezizaene, cis-muurola-4(15),5-diene, β-acoradiene, α-neocallitropsene and acorenol, preferably consisting of α-funebrene, β-funebrene, unknown terpene Y and β-acoradiene, more preferably consisting of α-funebrene and β-acoradiene are contained in the zizaene-containing composition provided in step a) and at least one, preferably at least two of said compounds are oxidized in step b).

30. An oxidized composition obtainable with the method of claim 26.

31. A non-human host cell expressing the polypeptide exhibiting zizaene synthase activity as characterized in claim 18 from the heterologous polynucleotide, wherein the non-human host cell is transgenic for the polynucleotide encoding said polypeptide.

32. A polypeptide exhibiting zizaene synthase activity and comprising an amino acid sequence which is less than 100%, but at least 85% identical to the amino acid sequence as shown in SEQ ID NO: 1 or a fragment thereof exhibiting zizaene synthase activity.


SEQUENCE LISTING (29 sequences)

SEQ ID NO: 1 - protein, 557 aa, Capsella rubella
MEAIRNLDQKNFFSFTELPPSQWGDYFLRVSITDLDFDVLEREIELLKPKVRENIFILSSSDKDATKRKILSIHFLESLGLSYHFDKEIEDSLKHAFENIEDLIADENDLYIISIMFRVFRTYGHNMLSDVFNRFKGSDGKFKETLIQDVKGMLSLYEAVHFGTTTDHIFDEASSFTLNNLEPLATGRRSCPPHISKLIRNALHIPQHRNIQALVAREYISFYEHEEDHDDTLLKLAKLNFKFLQLHYFQELKTITMWWRELDHTLNFPLSFRERTVEAWFSALTMYFEPQFSVGRTLSAKLYLVLTFLDDACDIYGSVPEVESLVNCLERWDPDYMENLQDHMKTAFKIVMYVFKEHEEILRSQGRLFALEKMIEEFKIVARKNLELLKWSRGGHDPSFDEYIESGGAEVATYATIASSILGLGEIGSKEAFEWLISRPKLVRTLGAQTRLLDDIADYEDDMEKGYIANALNYYMKEHGLTKDEASRELEKMNEDMNKIINEECLQLTTMPRRVLMQYVNFRRLLDVLYTNDDVYNHREGKLKEYISLLLVDPILL

SEQ ID NO: 2 - DNA, 1671 bp, Capsella rubella
  source 1..1671: mol_type = genomic DNA
atggaagcgatccgtaacctggaccagaaaaacttcttcagcttcactgaactgccgccgagccagtggggtgactacttcctgcgtgtttccatcactgacctggacttcgacgttctggaacgtgaaatcgagctgctgaaaccgaaagttcgcgaaaacatcttcatcctttcttcttccgacaaagacgcgaccaaacgtaaaatcctgtctatccacttcctggaaagcctgggtctgtcttaccactttgataaagaaatcgaagatagcctgaaacacgcgttcgaaaacatcgaggacctgatcgctgacgaaaacgacctgtacatcatctccatcatgttccgtgttttccgtacctacggccacaacatgctgagcgacgtattcaaccgcttcaaaggttccgacggtaaattcaaagaaaccctgatccaggacgtgaaaggtatgctgtctctgtacgaagcggtgcactttggcaccactaccgaccacatcttcgacgaggcgtccagcttcaccctgaacaacctggaaccgctggctactggccgtcgttcttgtcctccgcacatctccaaactgatccgtaacgcgctgcacatcccgcagcaccgcaacattcaggcgctggttgcgcgcgaatacatttccttctacgaacacgaagaggaccacgacgacaccctgctgaagctggcaaaactgaacttcaaattcctgcagctgcactatttccaggaactgaaaactatcaccatgtggtggcgcgaactggatcacaccctgaacttcccgctgtctttccgcgaacgtactgtggaagcctggttcagcgcgctgactatgtacttcgaaccgcagttcagcgtgggccgtaccctgtccgcaaaactgtacctggttttgaccttcctggacgacgcgtgcgacatctacggctctgtgccggaagtagaatctctggtgaactgcctggaacgctgggacccggactacatggaaaacctgcaggaccacatgaaaaccgcattcaaaatcgttatgtacgtgtttaaagaacacgaagaaatcctgcgttcccagggccgcctgttcgcgctggaaaaaatgatcgaagaatttaaaatcgtggcacgtaaaaacctggaactgctgaaatggtctcgtggtggtcacgacccgtccttcgacgaatacatcgaaagcggcggcgcggaagttgctacctacgcgaccattgcctcttctatcctgggcctgggtgaaatcggttccaaagaagcgttcgagtggctgatctcccgcccgaaactggttcgtaccctgggtgcgcagacccgcctgctggacgacattgcggactacgaggacgatatggaaaaaggttacatcgcgaacgcgctgaactactacatgaaagaacacggcctgaccaaagacgaggcgagccgtgaacttgaaaaaatgaacgaagatatgaacaaaatcatcaacgaagaatgtctgcagctgaccaccatgccgcgtcgcgttctgatgcagtacgttaacttccgccgtctgctggacgttctgtacaccaacgacgacgtttacaaccaccgtgaaggtaaactgaaagaatacatctccctgctgctggttgatccgatcctgctg

SEQ ID NO: 3 - DNA, 1689 bp, synthetic construct
  source 1..1689: mol_type = other DNA
  misc_difference 1..7: note = linker
  misc_difference 1679..1689: note = linker
ggatccgatggaagcgatccgtaacctggaccagaaaaacttcttcagcttcactgaactgccgccgagccagtggggtgactacttcctgcgtgtttccatcactgacctggacttcgacgttctggaacgtgaaatcgagctgctgaaaccgaaagttcgcgaaaacatcttcatcctttcttcttccgacaaagacgcgaccaaacgtaaaatcctgtctatccacttcctggaaagcctgggtctgtcttaccactttgataaagaaatcgaagatagcctgaaacacgcgttcgaaaacatcgaggacctgatcgctgacgaaaacgacctgtacatcatctccatcatgttccgtgttttccgtacctacggccacaacatgctgagcgacgtattcaaccgcttcaaaggttccgacggtaaattcaaagaaaccctgatccaggacgtgaaaggtatgctgtctctgtacgaagcggtgcactttggcaccactaccgaccacatcttcgacgaggcgtccagcttcaccctgaacaacctggaaccgctggctactggccgtcgttcttgtcctccgcacatctccaaactgatccgtaacgcgctgcacatcccgcagcaccgcaacattcaggcgctggttgcgcgcgaatacatttccttctacgaacacgaagaggaccacgacgacaccctgctgaagctggcaaaactgaacttcaaattcctgcagctgcactatttccaggaactgaaaactatcaccatgtggtggcgcgaactggatcacaccctgaacttcccgctgtctttccgcgaacgtactgtggaagcctggttcagcgcgctgactatgtacttcgaaccgcagttcagcgtgggccgtaccctgtccgcaaaactgtacctggttttgaccttcctggacgacgcgtgcgacatctacggctctgtgccggaagtagaatctctggtgaactgcctggaacgctgggacccggactacatggaaaacctgcaggaccacatgaaaaccgcattcaaaatcgttatgtacgtgtttaaagaacacgaagaaatcctgcgttcccagggccgcctgttcgcgctggaaaaaatgatcgaagaatttaaaatcgtggcacgtaaaaacctggaactgctgaaatggtctcgtggtggtcacgacccgtccttcgacgaatacatcgaaagcggcggcgcggaagttgctacctacgcgaccattgcctcttctatcctgggcctgggtgaaatcggttccaaagaagcgttcgagtggctgatctcccgcccgaaactggttcgtaccctgggtgcgcagacccgcctgctggacgacattgcggactacgaggacgatatggaaaaaggttacatcgcgaacgcgctgaactactacatgaaagaacacggcctgaccaaagacgaggcgagccgtgaacttgaaaaaatgaacgaagatatgaacaaaatcatcaacgaagaatgtctgcagctgaccaccatgccgcgtcgcgttctgatgcagtacgttaacttccgccgtctgctggacgttctgtacaccaacgacgacgtttacaaccaccgtgaaggtaaactgaaagaatacatctccctgctgctggttgatccgatcctgctgtaagcggccgc

SEQ ID NO: 4 - protein, 555 aa, Vetiveria zizanoides
MATTAAFCLTTTPIGEPVCRRQYLPTVWGSFFLTYQPCTPEEVQSMEERALAKKTEVGRMLQEVAASSNLARKLGLVDELERLGVDYHYKTEINDLLGAIYNGKDDDNGGSDDDLYITSLKFYLLRKHGYALSSDVFLKFRDEQGNISSDDVKCLIMLYDASHLRIHEEKILDNINSFTKSCLQSVLETNLEPALQEEVRCTLETPRFRRVERIEAKRFISAYEKNIARDDALLEFARLDYNIVQILYCKELKELTVWWKEFHSRTNLTFARDRIVEMYFWVMAIIYEPCYSYSRIWVTKMFLSVALLDDIYDNYTSTEESNIFTTAMERWDVKATEQLPANMRTFYDYLICTTDEVVEELKLQNNKNAELVKKVLIDAAKCYHSEVKWRDDHYVPNDVGEHLQLSMRSIAAMHSINFVFISLGAVCTREAVECAFTYPKIIRGICVHARISNDIASHEREQASEHMASTLQTCMKQYGITVEEAAEKLRVINEESWMDIVEECLYKDQYPLALSERVVAFAQSICFMYNGVDKYTIPSKLKDSLDSLYVNLIPV

SEQ ID NO: 5 - DNA, 1674 bp, synthetic construct
  source 1..1674: mol_type = other DNA
atggaggcgatccgcaacctggatcagaagaacttcttctcctttaccgagctgccgccctcgcagtggggcgactatttcctccgggtgtcgatcactgacctcgatttcgacgtgctcgagcgggagattgagctcctgaagcccaaggtgcgcgagaatatcttcatcctctcgagctcggacaaggatgcgacgaagcgcaagatcctgtcgatccatttcctggaaagcctgggactcagttaccatttcgacaaggagatcgaggactccctgaagcatgcgttcgagaacatcgaggacctcatcgccgacgagaacgacctctacatcatctcgatcatgttcagggtgttccgaacctatggtcacaacatgctctccgatgttttcaaccgcttcaaaggctccgacggcaagttcaaggagacgctcatccaggatgtgaaggggatgctgagcctctacgaggccgtccacttcggcaccacgaccgaccatattttcgacgaggcttcgtcgttcaccctgaacaacctggagccgctcgcgaccgggcggcgctcctgcccgccgcacatctcgaagctgatccgcaatgcgctccatatcccgcagcatcggaacatacaggcgctggtggcccgcgaatatatctcgttctacgagcacgaggaagatcacgacgacacgctgctcaagctcgccaagttgaatttcaagttcctgcagctgcactatttccaggagctgaagaccatcacgatgtggtggcgcgaactggaccatacgttgaattttcccctttcctttcgggagcggacggtcgaggcctggttctcggcgctgaccatgtatttcgagccccaattctcggtgggccgcacactgtctgcgaagctctatctggtgctgaccttccttgacgacgcctgcgacatctacgggagcgtgcccgaggtggagagcctggtcaactgcctcgagcgctgggaccccgactacatggagaatctacaggaccacatgaagacggccttcaagatcgtcatgtatgtcttcaaggagcatgaggaaatcctgcgctcgcagggccggctcttcgccctcgagaagatgatcgaagagttcaagatcgtggcgcgcaagaacctggagctgcttaaatggtcgcgcggcgggcacgacccttcattcgatgaatacatcgagagcggcggcgccgaggtcgcaacctacgccacgatcgcgagctccatcctggggctgggcgagatcggcagcaaggaggcgttcgaatggctgatcagccggccgaagctcgtgcggaccctcggcgcgcagacgcgtcttctggatgacatcgccgattacgaggatgacatggagaagggctacatcgcgaacgccctgaactattacatgaaagagcatggcctcaccaaggacgaagcctcgcgcgagctcgagaaaatgaacgaggacatgaacaagatcattaacgaggaatgcctgcagcttaccaccatgccgagacgcgtcctgatgcagtatgtgaacttccgccggctgctcgacgtgctgtatacgaacgatgatgtctacaaccaccgcgagggcaagctgaaggaatatatctcgcttctgctcgtcgacccgatcctcctctga

SEQ ID NO: 6 - DNA, 1668 bp, synthetic construct
  source 1..1668: mol_type = other DNA
atggccaccaccgccgccttctgcctcaccaccaccccgatcggcgagccggtgtgccgccgccagtacctcccgaccgtgtggggctcgttcttcctcacctaccagccgtgcaccccggaggaggtgcagtcgatggaggagcgcgccctcgccaagaagaccgaggtgggccgcatgctccaggaggtggccgcctcgtcgaacctcgcccgcaagctcggcctcgtggacgagctcgagcgcctcggcgtggactaccactacaagaccgagatcaacgacctcctcggcgccatctacaacggcaaggacgacgacaacggcggctcggacgacgacctctacatcacctcgctcaagttctacctcctccgcaagcacggctacgccctctcgtcggacgtgttcctcaagttccgcgacgagcagggcaacatctcgtcggacgacgtgaagtgcctcatcatgctctacgacgcctcgcacctccgcatccacgaggagaagatcctcgacaacatcaactcgttcaccaagtcgtgcctccagtcggtgctcgagacgaacctcgagccggccctccaggaggaggtgcgctgcaccctcgagacgccgcgcttccgccgcgtggagcgcatcgaggccaagcgcttcatctcggcctacgagaagaacatcgcccgcgacgacgccctcctcgagttcgcccgcctcgactacaacatcgtgcagatcctctactgcaaggagctcaaggagctcaccgtgtggtggaaggagttccactcgcgcaccaacctcaccttcgcccgcgaccgcatcgtggagatgtacttctgggtgatggccatcatctacgagccgtgctactcgtactcgcgcatctgggtgaccaagatgttcctctcggtggccctcctcgacgacatctacgacaactacacctcgaccgaggagtcgaacatcttcaccaccgccatggagcgctgggacgtgaaggccaccgagcagctcccggccaacatgcgcaccttctacgactacctcatctgcaccaccgacgaggtggtggaggagctcaagctccagaacaacaagaacgccgagctcgtgaagaaggtgctcatcgacgccgccaagtgctaccactcggaggtgaagtggcgcgacgaccactacgtgccgaacgacgtgggcgagcacctccagctctcgatgcgctcgatcgccgccatgcactcgatcaacttcgtgttcatctcgctcggcgccgtgtgcacccgcgaggccgtggagtgcgccttcacctacccgaagatcatccgcggcatctgcgtgcacgcccgcatctcgaacgacatcgcctcgcacgagcgcgagcaggcctcggagcacatggcctcgaccctccagacctgcatgaagcagtacggcatcaccgtggaggaggccgccgagaagctccgcgtgatcaacgaggagtcgtggatggacatcgtggaggagtgcctctacaaggaccagtacccgctcgccctctcggagcgcgtggtggccttcgcccagtcgatctgcttcatgtacaacggcgtggacaagtacaccatcccgtcgaagctcaaggactcgctcgactcgctctacgtgaacctcatcccggtgtga

SEQ ID NO: 7 - protein, 557 aa, synthetic construct
MEAIRNLDQKNFFSYTELPPSQWGDYFLRVTISDLDYEVIEREVELLKPKVRENIFILSSSDKDASKRKILSIHFLESLGLSYHFDKEIEDSLKHAYENIEDLIADENDLYIISIMFRVFRTYGHNMLSDVFNRFKGSDGKFKETLIQDVKGMLSLYEAVHWGTTTDHIFDEASSFTLNNLDPLATGRRSCPPHITKLIMNALHIPNHRNIQALVAREYISFYEHEEDHDDTLLRLAKLNFKFLQLHYFQELKTITMWWRELDHTLNFPLSFRERTVEAWFSGLTMFFDPQFSVGRTISAKLYLVLTFLDDACDIYGSVPEVESLVNCLERWDPDYMENLQDHMKTAFKVVMYVFKEHEEILRSQGRLFALEKMIEEFKIVARKNLELLKWSRGGHDPSFDEYIESGGAEVATYATIASSILGLGEIGSKEAFEWLISRPKLVRTLGAQTRLLDDIADYEDDMEKGYIANALQYYMMDHGLTKDEATRELDKMNEDMNKIVNEECIQLTTMPRRVLMQYVNYRRLLDVLYTNDDVYNHREGKLKEYISLLLVDPILL

SEQ ID NO: 8 - protein, 557 aa, synthetic construct
MEAIRNLDQKNFFTFTELPPSQWGDYFLKVSITDLDFDVLEREIELLKPKVRENIWILSSSDKDATKRKIVSIHFLESLGLSYHFDKEIEDSLKHAFENIEDLIADENELYIISIMFRVFRTYGHNMLSDVFNRFKGSDGMFKETLLQDVKGMLSLYEAVHFGTTTDHIFDEASSFTLNNLEPLATGRRSCPPHISMLIRNALHIPQHRNIQALVAREYISFYEHEEDHDDTLLKLAKLNFKFLQLHYFQELKTITMWWRELDHTLNFPISFRERTVEAWFSALTMYFEPQFSLGRTLSAKLYLVLTFLDDGCDIYGSVPEVDSLVNCLERWDPDYMENLQDHMKTAFKILMYVFKEHEEILRSQGKLFALEKMLEEFKIVGRMNLELLKWSRGGHDPSFDEYIESGGAEIATWATIASTILGLGEIGSKEAFEYLISRPKIVRTLGAQTRLLDDIADYEDDMEKGYIAQALNYYMKEHGLTKDEASRELDKMNEDKNKIINEECLQVTTMPRRVLMQYVNFRRLLDVLYTNDDVYNHKEGKLMEYISLLLVDPILL

SEQ ID NO: 9 - protein, 557 aa, synthetic construct
MEALKNLDQKNWFSFTEIPPSQWGDYFLRITVTDLDFDVLERDIELLKPKVRENIFILSTSDKDATKRKILTIHFLESLGLSFHFDKEIEDSLKHGFEQIEDLVAEENDLYIISIMFRVFRTYGHNMLSDVFNRFKGSDGKFKETLIQDVKGMLSLYEAVHFGTTTDHIFEEASSFTLNNLEPVATGMRSCPPHISKLIRNALHIPQHKNIQAVVAREYISYYDHEEDHDDTLLKLAKLNFKFVQLHYYQELKSITKWWRELDHTLNFPLSFRDKTLEAWFSALTMWFEPQFSLGRTLSAKLYLVLSFVDDGCDIYGSVPEVESLVNCLERWDPDFMENLQDHMKTAFKIVMFVFKEHDEVLMTQGRLFALEKMIEEWKIVARKNLDLLKWSRGAHDPSFDEYIESGGAEVATYATIASSILGIGDIGSKEAFEWLISRPKLVRTIGAQTRLLDDIAEFEDDMEKGYIANALNYYMKEHALTKDEASREVEKMNEDMNKLINEECLQVTTKPRRVLMQYVNFRRLLDVLYTNDDVYNHREGKLKEFISLLIVDPIVL

SEQ ID NO: 10 - protein, 557 aa, synthetic construct
MDAIRNIDQMNFFSFTDIPPSQWGDYFLRISLTDIDFDVVEREIELLKPKVRENIFILSSSDMDATKKKIVSIHWLESLGLSYHFDKEIEESLKHAFENIEDLLADENDLYIISIMFRVFRTYGHNMLSDVFNRFKASDGKFKETLIQDVKGMLSLYEAVHFGTTTDHIFDEASSFTLQNLEPLATGRRSCPPHISKVIRQGLHIPQHRNIQAIVAREYISFYEHEEDHDDTLLKLAKLNFKFLQLHYFQELKTITMWWRELDHTVNFPLSFREMTVDAWFTAVTMYWEPQWSVGRTVSAKLYLVLTWLDDACDIYGSVPEVESLVNCVERWDPDFMENLQDHMMTAFKIVMYVFMEHEEILRSNGRLFGLEKMIEEFKLVARMNLELLKWSRGGHDPTFDEYVESGGAEVATYATIGSSILGLGEIGSKEAWEWLISRPRLVRTLGGQSRLLDDLADYEDDMEKGFIANALNYYMKEHGLTKDEASRELEKMNDEMNKIINEECLQLTTMPMRVVMQFVNWRRLLDILYTNDDVYNHRDGKLKEYISLLLLDPILL

SEQ ID NO: 11 - protein, 557 aa, synthetic construct
MDGIRNLDNKNFFTWTDLPPSQWGDWFIRISISDLDFDVLEREIELLKPKVRENIFIISSTDKDATKRMILSIHFLESLGLSYHFDREIEDSLKHAFENIEDIIADENDLYIVSIMFRVFRTFGHNRLSDVFNMFMGSDGKFMESVIQDVKGMLTLYEAVHYATTTDHIFDEASSFTLNNLEPLGTGKRSCPPHISKVIRNALHIPQHRNIQALVAREYISFYEHEEDHDDTLLKLAKLNFKFLQLHYFQELKTITKWWRELDHTLNFPLSYRERTVEAWFSAITMYWEPQFSVGRTLSAKVFLVLTFIDDGCDLYGSVPEVESLVNCLERWDPDFMENLQDHMKTAFKIVKWVFKEHEEIVRSQGRVFALERMIEEWKLVARKNVELLRWSMGGHDPSYEEYVETGGGEVATYATIGSTLLGLGEIGSMEAFEWLLTKPRLIRTIGAQTRLLDDIADYEDDMEKGYIANALNYYMKEHGLTKDDASMEVEKKNEDMNKIINEECVQLTSKPRMVVMQWVNYMRVLDVLYTNDDVYNHREGKVKEYLTILVVDPIII

SEQ ID NO: 12 - protein, 557 aa, synthetic construct
MEAIRNLDQMNFFSFTELPPTNWGDYFLMVSITDLDWDVLERDLELLKPKVRENIFIVSSSDKDATMMKILSIHFLESLGLSYHFDKEIEDSLKHAFENIEEVIGDENDLFIISIMFRVFRTYGHQRLSDVFNRFMGTDGKFKESLLNDVKGRLSLYEAVHFGSTTDHLFDDATSFTLNQLEPLATGRRSCPPHISKLIMNALHVPNHRQIQALVAMDFISFYEHEEDHDDTLLKLAKLNFKFLQLHYYQELKTITMWWMELDHTLNYPLSWREMTIEAWFSALTMYFEPQFSVARSLSAKLYLILTYLDDACDIYGSVPEVESLVNCLERWDPDWMENLQDHMKTAFKILKYVFKEHEEIVKSQGKLFGVDKMIDEFKIVAMMNIELLKWSRAGHDPSFDEYVESGGAEVATYATLASSILGLGEIGSMEAFEWLITRPRLLRTIGAQTRLIDDIADYEDDMEKGYIANALNYYMKEHGISRDEASREIEKKNDDMNKIINEECLQITTKPRRVVRQYVNFRRVLEVLYSNDDIYQHREGKLREYITLLLVEPILL

SEQ ID NO: 13 - protein, 563 aa, Capsella rubella
MEEVKNLDQGSFLNFTKLPPSQWGDQFLKFSKADSDFDVLEREIEVLKPKVRENIFMLSSRDKDATKKTIHSIHLLISLGLSYHFENEIEETMKHAFEKIEDLLADENDLYTISIMFRVFRTYGHNMLSDVFNRFKGNDGKFKETLLQDVKGMLSLYEAVHFGTTTDHILDEASSFTLNHLEPLATGRKSCPPHISKLIRNALHIPQHRNIQALVAREYISFYEHEEDREDTLLKLAKLNFKFLQLHYFQELKTITMWWRELDHTSNLPANFRERTIETWFAALMMYFEPQFSLGRIMSAKLYLVITFLDDACDIYGSIPEVESLVDCLERWDPDYMENLEGHMKTAFKFVMYLFKEYEEILRSQGRLFVLEKMIEEFKIIARKNLELVKWARGGHVTSFDEYVESGGAEIGTYATIACSFMGIGEIGRKEAFEWLISRPKLVRTLGAKTRVMDDIADYEEDMEKGYTANALNYYMKEHKITKEEASRELEKVVRDMNKIVNEECLKMTAMPRRILMQSVNYGRSLDVLYTADDVYNNREGKLKEYISLVLVDPIRSYTSLET

SEQ ID NO: 14 - protein, 460 aa, Capsella rubella
MEEVKNLDQESFLNFTKLPASQWGDQFLKFSKADSDFDVLEREIEVLKPKVRENIFMLSSKDKDTTKRTIHSIYLLESLGLAYHFDKEIEDSLKHAFEKIEDLIADEDDLYTISIMFRVFRTYCHNMLSDVFNRFKGKDGKFKETLIQDVKGILSLYEAVQFGTTTDHILDEASSFTLNHLEPLATGRKSCSPHILKLIRNAFHIPQHRNNQALVAREYISFYEHEEDRDDTLLKLAKVNYKFLQLHYFQELKTITMWWRNLEHTANLPPNFRERTVETWFSALLMYFEPQFSLGRIMSAKMYLVITFLDDACDIYGSIPEVESLVDCLERWDPDYMENLQGHMKTAFKFVMYLFKEYEEILRSQGRLFVLEKMIEEFKILARTNLELVKWARAGHVPSLDEFLESGGAEIATYATIACSIMGLGEMGSKEDFDWLISRPKLARTLGAKTRLMDDIADFE

SEQ ID NO: 15 - protein, 557 aa, Arabidopsis thaliana
MEALGNFDYESYTNFTKLPSSQWGDQFLKFSIADSDFDVLEREIEVLKPKVRENIFVSSSTDKDAMKKTILSIHFLDSLGLSYHFEKEIEESLKHAFEKIEDLIADENKLHTISTIFRVFRTYGYYMSSDVFKIFKGDDGKFKESLIEDVKGMLSFYEAVHFGTTTDHILDEALSFTLNHLESLATGRRASPPHISKLIQNALHIPQHRNIQALVAREYISFYEHEEDHDETLLKLAKLNFKFLQLHYFQELKTITMWWTKLDHTSNLPPNFRERTVETWFAALMMYFEPQFSLGRIMSAKLYLVITFLDDACDTYGSISEVESLADCLERWDPDYMENLQGHMKTAFKFVMYLFKEYEEILRSQGRSFVLEKMIEEFKIIARKNLELVKWARGGHVPSFDEYIESGGAEIGTYATIACSIMGLGEIGKKEAFEWLISRPKLVRILGAKTRLMDDIADFEEDMEKGYTANALNYYMNEHGVTKEEASRELEKMNGDMNKIVNEECLKITTMPRRILMQSVNYARSLDVLYTADDVYNHREGKLKEYMRLLLVDPILL

SEQ ID NO: 16 - protein, 557 aa, Arabidopsis thaliana
MEALGNFDYESYTNFTKLPSSQWGDQFLKFSIADSDFDVLEREIEVLKPKVRENIFVSSSTDKDAMKKTILSIHFLDSLGLSYHFEKEIEESLKHAFEKIEDLIADENKLHTISTIFRVFRTYGYYMSSDVFKIFKGDDGKFKESLIEDVKGMLSFYEAVHFRTTTDHILDEALSFTLNHLESLATGRRASPPHISKLIQNALHIPQHRNIQALVAREYISFYEHEEDHDETLLKLAKLNFKFLQLHYFQELKTITMWWTELDHTSNLPPNFRERTVETWFAALMMYFEPQFSLGRIMSAKLYLVITFLDDACDTYGSISEVESLADCLERWDPDYMENLQGHMKTAFKFVMYLFKEYEEILRSQGRSFVLEKMIEEFKIIARKNLELVKWARGGHVPSFDEYIESGGAEIGTYATIACSIMGLGEIGKKEAFEWLISRPKLVRILGAKTRLMDDIADFEEDMEKGYTANALNYYMNEHGVTKEEASRELEKMNGDMNKIVNEECLKITTMPRRILMQSVNYARSLDVLYTADDVYNHREGKLKEYMRLLLVDPILL

SEQ ID NO: 17 - protein, 557 aa, Arabidopsis lyrata subsp. lyrata
MDEVKNLDRKSFLDFTKLPPSQWGDQFLKFSMADSDFDALEREIKVLKPKVRENIFILSSRDKDAMKRTILSIHFLNSLGLSYHFEKEIEESLKHAFDEVEDLIADENDLYTISTIFRVFRTYGYDMSSDVFNKFKGDDGKFKEILIEDVRGMLSFYEAVHFGTTKDHILDETSCFTLNHLEPLATGRRVCPPHISKLIQNAIHIPQHRNSQALVAREYISFYEHEEDRDEILFKLAKLNFKFLQLHYFQELKTITMWWRELDHTSNFPPNFRERTVETWFAALMMYFEPQFSLGRIMSAKLYLVITFLDDACDIHGSIPDVESLVDCLERWDPDYMENLHGHMKTAFKFVMYLFEEYEEILRSQGRSFVLEKMIEEFKIIARKNLELVKWARGGHVPSFDEYIESGGAEIGTYATIACSIMGLGEIGKKEAFEWLISRPKLVRTLGAKTRLMDDIADFKEDMEKGYTANAVNYYMNEHGVTKEEANRELEKMNGDMNKIVNEECLKISTMPRRILMQSVNYGRSLDVLYTADDVYNHREGKLQEYISLLLVDPILL

SEQ ID NO: 18 - protein, 557 aa, Arabidopsis thaliana
MEALGNFDYESYTNFTKLPSSQWGDQFLKFSIADSDFDVLEREIEVLKPKVRENIFVSSSTDKDAMKKTILSIHFLDSLGLSYHFEKEIEESLKHAFEKIEDLIADENKLHTISTIFRVFRTYGYYMSSDVFKIFKGDDGKFKESLIEDVKGMLSFYEAVHFGTTTDHILDEALSFTLNHLESLATGRRASPSHISKLIQNALHIPQHRNIQALVAREYISFYEHEEDHDETLLKLAKLNFKFLQLHYFQELKTITMWWTKLDHTSNLPPNFRERTVETWFAALMMYFEPQFSLGRIMSAKLYLVITFLDDACDTYGSISEVESLADCLERWDPDYMENLQGHMKTAFKFVMYLFKEYEEILRSQGRSFVLEKMIEEFKIIARKNLELVKWARGGHVPSFDEYIESGGAEIGTYATIACSIMGLGEIGKKEAFEWLISRPKLVRILGAKTRLMDDIADFEEDMEKGYTANALNYYMNEHGVTKEEASRELEKMNGDMNKIVNEECLKITTMPRRILMQSVNYARSLDVLYTADDVHNHREGKLKEYMRLLLVDPILL

SEQ ID NO: 19 - protein, 557 aa, synthetic construct
MEAVMNIDQKNFFTFTEIPPSQWAEYFLRLSISDLEFDILDREIELLKPKVRENIFILSSSDKEATRRMILSIHFLESLGLTYHFDKEIEDTLRHAWENLEDLVADENELYIISIMFRVFRTYGHNMLSDVFQKFRGTDGMFMETVIQDVRGMLSLYEAVHWATTTDHLFDEATSFTLNNLEPIGTGRRSCPPHISRIIRNALHIPQHRNVQAVVGKDYISFYEHDEEHEDSLLKLAMLNFKFLQLHYWQELRTITMWWMEVDHTINFPLSFRERTVEAWFSAVSMWFEPQFSVARTISAKLWLVISYIDDACDIWGSVPEVDTLLNCLDRWDPDWMENLQDHKKTAFKIVMYVWKEHEEILKSQAMLWAVEKMIEEFKIVARKNLELLKWTKGGHEPTFDEYVDSAGAEIASYATVGSTILGLGEIASKEAFEFILSRPKIVRTIGAQTRLLDDIGEYEDDMEKGYIANALNYYMMEHGITMDEASRDLEKMNEEMNKVLNEDCIQLTTMPRRVLMNYVNYRRLLDVLYTNDDVYNHRDGKIKEFISLVLVDPVLL

SEQ ID NO: 20 - protein, 557 aa, synthetic construct
MEAIMNIEQKNFFSWSEVPPSNWGDYFLRISITDLDFEVIERELELLKPMVRENIWVLTSTDREATKRKILSIHYLESLGLSFHFDKEIEDSLKHAYENIDELIADENDLYIISIMFRVFRTYGHNRLSDVFNRFKGSDGKFKETLIQDVKGKLSLYEAVHWASSTDHIFDEASSFTINNLEPIATGKMSCPPHLSMIIRQALHIPQHKNLQALVGREYISFYDHEDDHEETLLKLAMVNFKFLQLHYFQELMTLTRWWRELDHSLNFPLSFRERTVEAWWTALTMYFEPQWSVGRTLSAKLWLLLSFVDDACDIFGSVPEVESLVNCLERWDPEWRDNLQDHMKTAFKVLMYVWKDHEEIVRSQGMLYGIEKKIEEFMLVARKNLELLRWSRGAHEPSFEEYIESAGAEIATYATLGSSILGLGEIGSKDGFDWLISMPRLVRTIAAQTRLLDDIGEYEDDRERGWIANGLNYYMREHGISRDEASRELERMNEEMNKVINEECLNLTTMPRKVVMQYVNYRRLLDVLYSQEDVYQHREGKIKEYISVLLVDPILL

SEQ ID NO: 21 - protein, 557 aa, synthetic construct
MEAVKNIDQMNYFSYTELPPSQWGEWFLMITITELDFEVIDREIEVLKPRVRENIWILSSSEKDATRMKILSIHFLDSLGLSFHFDREIDETLRHGFEQIDDILAEENDLYIISIMFRVFRTFGHNMLSDVFNKFKGTDGMFKETIINDVKGKLSLYEAVHFGTSTDHIFDEASSFSIQNLEPLATGMRTCPPHISKLIKNALHLPQHRNLQAVVAREYITFYEHDEDHEDTLLMLAKLNFKWLQVHYYQELRTITKWWRDVDHTLNWPLSFRDMTVEGWFSALTMYFEPNFSVGRTITAKVYVVITFLDDGCDIWGSLPEVESLVQCLERWDPEWRENLQDHMKTGFMLIMYVFKEHEELLRSQGRIYAVDMMIDEFRLVGRKNIELVRWSRGGHDPTYDEYIESAGGEVGTWATIATTIIGLGDVGSKEAWDYIISKPKIVKSVGAQTRLLDDIADFDDDMERGYIANALNYYMREHGLTKDEASMELDRMQDDMNKIINDECIQLSTMPMRVLMQYLNYKRLIEVLYTNDDLYNHREGKVREYITLLLLDPILI

SEQ ID NO: 22 - protein, 556 aa, Brassica rapa subsp. pekinensis
MEEVKRDQKSYLNFSKLLPSQWGDHFLNISIADSDFDVLAREMEVLTPKLRKNIFTFSSRDKDAMKRSILSIHILDSLGLAYIFEKEIVETLKHAFEKIDELITDENDLYTVSIMFRVFRIYGHNMLSDVFDRFKRNDGKFKESLMEDVQGMLSFYEAAHFRTTTDHILDEALSFTLDYLESLATDSKAIPPHILKHIQNALYIPQHQNVQVLVAREYLSFYEQEEDNDETLLKLAKLNFKFLQLHYIQELKIITTWWRELDHTKNLPPGFRERTFECWFVGSMMYFEPQFSLGRIMSAKFFLLFTFLDDVCDTYGSIPEVESVVNCLERWDPEYMENLHGHMKTAFKFVMSVYKEFEEILKSQGRSFALEKMIEEFKIVARTNLDLIKWARAGQIPNFDEYVEAGGAEAGSYATIACSIMGLGEIGKKEDFEWLLSRPKSVRYLARKARLLDDITDFEEDMNKGYTANALNYYMKQHGVTKKEASREFHKMIGDINKLVNEECLKTTNISRPILMQVVNFGRMVNILYTSDDLYNHREGKLKDYLTALLVDPIHL

SEQ ID NO: 23 - protein, 559 aa, Eutrema salsugineum
MEAVRNILDQLQSSLNFSELPPSQWADKLLSVSIDDSVFDALAREIEVLKPKVRENVFMFSSRDKDAIKRNIISIHFIDSLGLSYYFEREIKETLKHAFQKIDDLIADENDMYTISIMFRVFRTYGHNISSDVFDRFKGNDGKFKKSLLEDVKGMLSFYEAVHFRTTTDDILDEALSFTMDHLESLAIDHKANPPHILKHIQNALYIPQHQKIQVLVAREYLSFYEQEDDHDDTLIKLAKLNFKFLQLLYIQELKTITTWWRGLDHTENLPSGFRERTFESWFVGAMMYFEPQFSLGRIMSAKFFLMFCFVDDACDTYGSILEVESVVDCLERWDPDYMENLQGHMKTAFKFVMYLYKEYEEILKSQGRSFALEKMIEEFKIVARTNLDLIKWARAGQIPNFDEYIEAGGAEAGSYATIACSIMGLGELGKKEDFEWLLSRPKSVRYLARKTRLLDDITDFEEDMNKGYTANALNYYMKQHGVTIEEATREFHKMIEVIEKVVNEECLKTTNISRPVLMQVVNFGRMVNILYTSDDVYNHREGKLKEYLTTLLVDPIHM

SEQ ID NO: 24 - protein, 556 aa, Brassica campestris
MEEVKRDQKSYLNFSKLLPSQWGDHFLNISIADSDFDVLAREMEVLTPKLRKNIFTFSSRDKDAMKRSILSIHILDSLGLAYIFEKEIVETLKHAFEKIDELITDENDLYTVSIMFRVFRIYGHNMLSDVFDRFIRNDGKFKESLMEDVQGMLSFYEAAHFRTTTDHILDEALSFTLDYLESLATDSKAIPPHILKHIQNALYIPQHQNVQVLVAREYLSFYEQEEDNDETLLKLAKLNCKFLQLHYIQELKIITTWWRELDHTKNLPPGFRERTFECWFVGSMMYFEPQFSLGRIMSAKFFLLFTFLDDACDTYGSIPEVESVVNCLERWDPEYMENLHGHMKTAFKFVMSVYKEFEEILKSQGRSFALEKMIEEFKIVARTNLDLIKWARAGQIPNFDEYVEAGGAEAGSYATIACSIMGLGEIGKKEDFEWLLSRPKSVRYLARKARLLDDITDFEEDMNKGYTANALNYYMKQHGVTKKEASREFHKMIGDINKLVNEECLKTTNISRSILMQVVNFGRMVNILYTSDDLYNHREGKLKDYLTALLVDPIHL

SEQ ID NO: 25 - protein, 443 aa, Noccaea caerulescens
MFRVFRRYGYNMSSDVFKRFKGNDGKFKDSLLEDVKGMLGFYEAVHFETTTDHILDEALSFTLNHLEPLATCCTTMPPHVSKLIQNSLHIPQNWNTPSLVAREYISFYEQEEDRDDTLLKLAKLNFNLLRLRYFQELKTITKWWREVSHSLNLPHHFKERTIEAWFSALMMYFEPKFSLERIITTKFIQVMTLFDHACDVYGSVPEAESLIDCLERRDPDYMENLQGHMKTVFNFVMCFFKDCGDVFKLLGKSFVLEMLIEEFMQYTRASLKLLKWVQGSHVPSFGEFMEVGGTVVGSEACVACCIMGLEETAGKEAFEWLRSRPKLVQALGAKLRILDDITDFEEDMGSGYTANGVNYYMNEHEVTKEEANRELEKMIGDINKIANEECLNMTTTMPHRNIMQAVRFGRAMDVLYIGDDVYNNRDGKLKEYMNSLLVDPIRY

SEQ ID NO: 26 - protein, 554 aa, Arabidopsis thaliana
MESQTKFDYESLAFTKLSHSQWTDYFLSVPIDDSELDAITKEIDIIKPEVRKLLSSKGDDETSKRKVLLIQSLLSLGLAFHFENEIKDILEDAFRKIDDITGDENDLSTISIMFRVFRTYGHNLSSNVFKRFTGDDGKFEQSLTEDAKGILSLYEAAHLGTTTDYILDEALKFTSSHLKSLLAGGTCRPHILRLIRNTLYLPQRWNMEAVIAREYISFYEQEEDHDKMLLRLAKLNFKLLQLHYIKELKTFIKWWMELDLTSKWPSQFRERIVEAWLAGLMMYYEPQFSGGRVIAAKFNYLLTILDDACDHYFSIPELTRLVDCVERWNHDGIHTLEDISQIIFKLALDVFDDIGRGVRSKGCSYYLKEMLEELKTLVRANLDLVKWARGNQLPSFEEHVEVGGIALTTYATLMYSFVGMGEAVGKEAYEWVRSRPRLIKSLAAKGRLMDDVTDFESDMSNGFAANAINYYMKQFVVTKEEAILECQKMVVDINKIVNEELLKTTTVPRRVLKQALNFGRLLEVLYTKSDDIYNCSEGKLKEYIVTLLIDPIHL

SEQ ID NO: 27 - protein, 554 aa, Arabidopsis thaliana
MESQTKFDYESLAFTKLSHSQWTDYFLSVPIDDSELDAITREIDIIKPEVRKLLSSKGDDETSKRKVLLIQSLLSLGLAFHFENEIKDILEDAFRRIDDITGDENDLSTISIMFRVFRTYGHNLPSSVFKRFTGDDGKFERSLTEDAKGILSLYEAAHLGTTTDYILDEALEFTSSHLKSLLVGGMCRPHILRLIRNTLYLPQRWNMEAVIAREYISFYEQEEDHDKMLLRLAKLNFKLLQLHYIKELKTFIKWWMELGLTSKWPSQFRERIVEAWLAGLMMYFEPQFSGGRVIAAKFNYLLTILDDACDHYFSIPELTRLVDCVERWNHDGIHTLEDISRIIFKLALDVFDDIGRGVRSKGCSYYLKEMLEELKILVRANLDLVKWARGNQLPSFEEHVEVGGIALTTYATLMYSFVGMGEAVGKEAYEWVRSRPRLIKSLAAKGRLMDDITDFESDMSNGFAANAINYYMKQFVVTKEEAILECQKMVVDINKIVNEELLKTTTVPRRVLKQALNFGRLLEVLYTKSDDIYNCSEGKLKEYIVTLLIDPIHL

SEQ ID NO: 28 - protein, 557 aa, Capsella bursa-pastoris
MEAIRNLDQKNFFSFTELPPSQWGDYFLRVSIADLDFDVLEREIELLKPKVRENIFMLSSSDKDAIKRKILSIHFLESLGLSYHFDKEIEDSLKHAFEKIEDLIADENDLYTISIMFRVFRTYGHNMLSDVFNRFKGNDGKFKESLIQDVKGMLSLYEAVHFGTTTDHIFDETSSFTLNNLEPLATGRRSCPPHISKLIRNALHIPQHRNIQALVAREYITFYEHEEDRDDTLLMLAKLNFKFLQLHYFQELKTITMWWRELDHTLNFPPSFRERTVEAWFSALTMYFEPQFSVGRTLSAKLYLVLTFLDDACDIYGSVPEVESLVNCLERWDPDYMENLQGHMKTAFKFVMYLFKEYEEILRSQGRLVVLEKMIEEVIILARKNLKLLKWSRGGHDPSFDEYIESGGAEVATYATIASSIMGLGEIGSKEAFEWLISRPKLVRTLGAKTRLLDDIADYEDDMERGYTANALNYYMKEHGLTKDEASRELEKMNEDMNKIINEEFLQLTTMPRRILMQYVNFRRLLDVLYTNDDVYNHREGKLKEYISLLLVYPIIL

SEQ ID NO: 29 - DNA, 1674 bp, synthetic construct
  source 1..1674: mol_type = other DNA
atggaggccatccgcaacctcgaccagaagaacttcttctcgttcaccgagctcccgccgtcgcagtggggcgactacttcctccgcgtgtcgatcgccgacctcgacttcgacgtgctcgagcgcgagatcgagctcctcaagccgaaggtgcgcgagaacatcttcatgctctcgtcgtcggacaaggacgccatcaagcgcaagatcctctcgatccacttcctcgagtcgctcggcctctcgtaccacttcgacaaggagatcgaggactcgctcaagcacgccttcgagaagatcgaggacctcatcgccgacgagaacgacctctacaccatctcgatcatgttccgcgtgttccgcacctacggccacaacatgctctcggacgtgttcaaccgcttcaagggcaacgacggcaagttcaaggagtcgctcatccaggacgtgaagggcatgctctcgctctacgaggccgtgcacttcggcaccaccaccgaccacatcttcgacgagacctcgtcgttcaccctcaacaacctcgagccgctcgccaccggccgccgctcgtgcccgccgcacatctcgaagctcatccgcaacgccctccacatcccgcagcaccgcaacatccaggccctcgtggcccgcgagtacatcaccttctacgagcacgaggaggaccgcgacgacaccctcctcatgctcgccaagctcaacttcaagttcctccagctccactacttccaggagctcaagaccatcaccatgtggtggcgcgagctcgaccacaccctcaacttcccgccgtcgttccgcgagcgcaccgtggaggcctggttctcggccctcaccatgtacttcgagccgcagttctcggtgggccgcaccctctcggccaagctctacctcgtgctcaccttcctcgacgacgcctgcgacatctacggctcggtgccggaggtggagtcgctcgtgaactgcctcgagcgctgggacccggactacatggagaacctccagggccacatgaagaccgccttcaagttcgtgatgtacctcttcaaggagtacgaggagatcctccgctcgcagggccgcctcgtggtgctcgagaagatgatcgaggaggtgatcatcctcgcccgcaagaacctcaagctcctcaagtggtcgcgcggcggccacgacccgtcgttcgacgagtacatcgagtcgggcggcgccgaggtggccacctacgccaccatcgcctcgtcgatcatgggcctcggcgagatcggctcgaaggaggccttcgagtggctcatctcgcgcccgaagctcgtgcgcaccctcggcgccaagacccgcctcctcgacgacatcgccgactacgaggacgacatggagcgcggctacaccgccaacgccctcaactactacatgaaggagcacggcctcaccaaggacgaggcctcgcgcgagctcgagaagatgaacgaggacatgaacaagatcatcaacgaggagttcctccagctcaccaccatgccgcgccgcatcctcatgcagtacgtgaacttccgccgcctcctcgacgtgctctacaccaacgacgacgtgtacaaccaccgcgagggcaagctcaaggagtacatctcgctcctcctcgtgtacccgatcatcctctga